SEQUENCE LISTING (41 sequences)

SEQ ID NO: 1 - protein, 9 aa, Infectious salmon anemia virus
HWKAAKYIK

SEQ ID NO: 2 - protein, 10 aa, Infectious salmon anemia virus
KEAVNRGHWK

SEQ ID NO: 3 - protein, 8 aa, Infectious salmon anemia virus
HKYNERLK

SEQ ID NO: 4 - protein, 8 aa, Infectious salmon anemia virus
KGYIWKHK

SEQ ID NO: 5 - protein, 10 aa, Infectious salmon anemia virus
KRMWDIGNKH

SEQ ID NO: 6 - protein, 14 aa, Infectious salmon anemia virus
KLIDEVEVIKKKKH

SEQ ID NO: 7 - protein, 17 aa, Infectious salmon anemia virus
HFRCMQGKQEVKGYIWK

SEQ ID NO: 8 - protein, 11 aa, Infectious salmon anemia virus
KTVHWHLRVVK

SEQ ID NO: 9 - protein, 10 aa, Infectious salmon anemia virus
KMTMMGKTVH

SEQ ID NO: 10 - protein, 12 aa, Infectious salmon anemia virus
KMGDTRKEGYCH

SEQ ID NO: 11 - protein, 23 aa, Infectious salmon anemia virus
KCWGMMFKTKSKMGDTRKEGYCH

SEQ ID NO: 12 - protein, 16 aa, Infectious salmon anemia virus
HAIIFGKGEDKSGQNK

SEQ ID NO: 13 - protein, 13 aa, Infectious salmon anemia virus
KVYGVLVDQLKLH

SEQ ID NO: 14 - protein, 7 aa, Infectious salmon anemia virus
KLHGKDK

SEQ ID NO: 15 - protein, 13 aa, Infectious salmon anemia virus
KLHGKDKVAGAKH

SEQ ID NO: 16 - protein, 9 aa, Infectious salmon anemia virus
KDKVAGAKH

SEQ ID NO: 17 - protein, 10 aa, Infectious salmon anemia virus
KQLHGQIHWK

SEQ ID NO: 18 - protein, 12 aa, Infectious salmon anemia virus
KFESPREFRKGH

SEQ ID NO: 19 - protein, 26 aa, Artificial Sequence
  Description of Artificial Sequence Synthetic peptide
HAQDILEKEHNGKLCSLKGVRPLILK

SEQ ID NO: 20 - protein, 30 aa, Artificial Sequence
  Description of Artificial Sequence Synthetic polypeptide
HAQDILEKEHNGKLCSLKGVRPXXXXLILK

SEQ ID NO: 21 - protein, 30 aa, Artificial Sequence
  Description of Artificial Sequence Synthetic polypeptide
HAQDXILEKEHNGKLCXSLKGVRXXPLILK

SEQ ID NO: 22 - protein, 19 aa, Artificial Sequence
  Description of Artificial Sequence Synthetic peptide
KEHNGKLCSLKGVRPLILK

SEQ ID NO: 23 - protein, 13 aa, Artificial Sequence
  Description of Artificial Sequence Synthetic peptide
KEHNGKLCSLKGK

SEQ ID NO: 24 - protein, 19 aa, Artificial Sequence
  Description of Artificial Sequence Synthetic peptide
XXXXXHXKXXKXXKXXXXX

SEQ ID NO: 25 - protein, 20 aa, Artificial Sequence
  Description of Artificial Sequence Synthetic peptide
XXXXXKXXXXXXHXKXXXXX

SEQ ID NO: 26 - protein, 18 aa, Artificial Sequence
  Description of Artificial Sequence Synthetic peptide
XXXXXHKXXXXXKXXXXX

SEQ ID NO: 27 - protein, 18 aa, Artificial Sequence
  Description of Artificial Sequence Synthetic peptide
XXXXXKXXXXKHKXXXXX

SEQ ID NO: 28 - protein, 20 aa, Artificial Sequence
  Description of Artificial Sequence Synthetic peptide
XXXXXKXXXXXXXKHXXXXX

SEQ ID NO: 29 - protein, 24 aa, Artificial Sequence
  Description of Artificial Sequence Synthetic peptide
XXXXXKXXXXXXXXKKKKHXXXXX

SEQ ID NO: 30 - protein, 27 aa, Artificial Sequence
  Description of Artificial Sequence Synthetic peptide
XXXXXHXXXXXXKXXXKXXXXKXXXXX

SEQ ID NO: 31 - protein, 21 aa, Artificial Sequence
  Description of Artificial Sequence Synthetic peptide
XXXXXKXXHXHXXXXKXXXXX

SEQ ID NO: 32 - protein, 20 aa, Artificial Sequence
  Description of Artificial Sequence Synthetic peptide
XXXXXKXXXXXKXXHXXXXX

SEQ ID NO: 33 - protein, 22 aa, Artificial Sequence
  Description of Artificial Sequence Synthetic peptide
XXXXXKXXXXXKXXXXHXXXXX

SEQ ID NO: 34 - protein, 33 aa, Artificial Sequence
  Description of Artificial Sequence Synthetic polypeptide
XXXXXKXXXXXXKXKXKXXXXXKXXXXHXXXXX

SEQ ID NO: 35 - protein, 26 aa, Artificial Sequence
  Description of Artificial Sequence Synthetic peptide
XXXXXHXXXXXKXXXKXXXXKXXXXX

SEQ ID NO: 36 - protein, 23 aa, Artificial Sequence
  Description of Artificial Sequence Synthetic peptide
XXXXXKXXXXXXXXXKXHXXXXX

SEQ ID NO: 37 - protein, 17 aa, Artificial Sequence
  Description of Artificial Sequence Synthetic peptide
XXXXXKXHXKXKXXXXX

SEQ ID NO: 38 - protein, 23 aa, Artificial Sequence
  Description of Artificial Sequence Synthetic peptide
XXXXXKXHXKXKXXXXKHXXXXX

SEQ ID NO: 39 - protein, 19 aa, Artificial Sequence
  Description of Artificial Sequence Synthetic peptide
XXXXXKXKXXXXKHXXXXX

SEQ ID NO: 40 - protein, 20 aa, Artificial Sequence
  Description of Artificial Sequence Synthetic peptide
XXXXXKXXHXXXHXKXXXXX

SEQ ID NO: 41 - protein, 22 aa, Artificial Sequence
  Description of Artificial Sequence Synthetic peptide
XXXXXKXXXXXXXXKXHXXXXX

(19) United States
(12) Patent Application Publication
(10) Pub. No.: US 2013/0243803 A1
(43) Pub. Date: Sep. 19, 2013

(54) THERAPIES, VACCINES, AND PREDICTIVE METHODS FOR INFECTIOUS SALMON ANEMIA VIRUS

(71) Applicants: Samuel Bogoch, New York, NY (US); Elenore S. Bogoch, New York, NY (US); Anne-Elenore Bogoch Borsanyi, New York, NY (US); Samuel Winston Bogoch, Oakland, CA (US)

(72) Inventors: Samuel Bogoch, New York, NY (US); Elenore S. Bogoch, New York, NY (US); Anne-Elenore Bogoch Borsanyi, New York, NY (US); Samuel Winston Bogoch, Oakland, CA (US)

(21) Appl. No.: 13/791,609

(22) Filed: Mar. 8, 2013

Related U.S. Application Data
(60) Provisional application No. 61/609,074, filed on Mar. 9, 2012.

(51) Int. Cl. A61K 39/145; C12Q 1/70; C07K 16/10

(52) U.S. Cl. CPC A61K 39/145; C07K 16/1018; C12Q 1/70
USPC 424/186.1; 530/328; 530/327; 530/326; 530/329; 530/387.9; 435/5

(57) ABSTRACT

The present invention provides therapies, vaccines, and predictive methods for infectious salmon anemia virus and provides compounds for diagnosing, preventing, and treating outbreaks of infectious salmon anemia virus including compounds for diagnosing, preventing, and treating infectious salmon anemia across different strains of virus.

This application claims priority to U.S. Provisional Application Ser. No. 61/609,074, filed Mar. 9, 2012, which is incorporated herein by reference in its entirety.

This application further incorporates by reference the following applications: U.S. Provisional Appln. Ser. No. 61/765,106, filed Feb. 15, 2013, U.S. Provisional Appln. Ser. No. 61/724,538, filed Nov. 9, 2012, U.S. application Ser. No. 13/553,137, filed Jul. 19, 2012, PCT/US2012/047451, filed Jul. 19, 2012, U.S. Provisional Appln. Ser. No. 61/509,896, filed Jul. 20, 2011, U.S. application Ser. No. 12/581,112, filed Oct. 16, 2009, U.S. Provisional Appln. Ser. No. 61/246,006, filed Sep. 25, 2009, U.S. application Ser. No. 12/538,027, filed Aug. 7, 2009, U.S. Provisional Appln. Ser. No. 61/185,160, filed Jun. 8, 2009, U.S. Provisional Appln. Ser. No. 61/179,686, filed May 19, 2009, U.S. Provisional Appln. Ser. No. 61/172,115, filed Apr. 23, 2009, U.S. application Ser. No. 12/429,044, filed Apr. 23, 2009, and PCT/US09/41565, filed Apr. 23, 2009, U.S. Provisional Appln. Ser. No. 61/143,618, filed Jan. 9, 2009, U.S. Provisional Appln. Ser. No. 61/087,354, filed Aug. 8, 2008, U.S. Provisional Appln. Ser. No. 61/054,010, filed May 16, 2008, U.S. application Ser. No. 12/108,458, filed Apr. 23, 2008, PCT/US2008/61336, filed Apr. 23, 2008, U.S. application Ser. No. 12/010,027, filed Jan. 18, 2008, U.S. Provisional Appln. Ser. No. 60/991,676, filed Nov. 30, 2007, U.S. application Ser. No. 11/923,559, filed Oct. 24, 2007, now U.S. Pat. No. 8,050,871, U.S. Provisional Appln. Ser. No. 60/982,336, filed Oct. 24, 2007, U.S. Provisional Appln. Ser. No. 60/982,333, filed Oct. 24, 2007, U.S. Provisional Appln. Ser. No. 60/982,338, filed Oct. 24, 2007, U.S. Provisional Appln. Ser. No. 60/935,816, filed Aug. 31, 2007, U.S. Provisional Appln. Ser. No. 60/935,499 filed Aug. 16, 2007, U.S. Provisional Appln. Ser. No. 60/954,743, filed Aug. 8, 2007, U.S. application Ser. No. 11/755,597, filed May 30, 2007, U.S. Provisional Appln. Ser. No. 60/898,097, filed Jan. 30, 2007, U.S. Provisional Appln. Ser. No. 60/880,966, filed Jan. 18, 2007, U.S. Provisional Appln. Ser. No. 60/853,744, filed Oct. 24, 2006, U.S. application Ser. No. 11/355,120, filed Feb. 16, 2006, U.S. application Ser. No. 11/116,203, filed Apr. 28, 2005, U.S. application Ser. No. 10/860,050, filed Jun. 4, 2004, now U.S. Pat. No. 7,442,761, U.S. application Ser. No. 10/189,437, filed Jul. 8, 2002, now U.S. Pat. No. 7,452,963, U.S. application Ser. No. 10/105,232, filed Mar. 26, 2002, now U.S. Pat. No. 7,189,800, U.S. application Ser. No. 09/984,057, filed Oct. 26, 2001, now U.S. Pat. No. 7,420,028, and U.S. application Ser. No. 09/984,056, filed Oct. 26, 2001, now U.S. Pat. No. 7,176,275, each in its entirety.

### SEQUENCE LISTING

The instant application contains a Sequence Listing, which has been submitted in ASCII format via EFS-Web and is hereby incorporated by reference in its entirety. Said ASCII copy, created on Mar. 8, 2013, is named 13794-48202_SL.txt and is 16,804 bytes in size.

### FIELD OF THE INVENTION

The present invention relates to therapies for preventing and treating infectious salmon anemia virus (ISAV) in salmon and other fish susceptible to ISAV, methods of differentiating lethality of ISAV and of predicting outbreaks of lethal ISAV, and compounds for diagnostic, therapeutic, and/or preventive purposes in ISAV.

### BACKGROUND OF THE INVENTION

Infectious salmon anemia virus (ISAV) is a virus of fish, particularly salmonids. The virus has been isolated from many different species of fish including Gadus morhua (Atlantic cod), Oncorhynchus kisutch (Coho salmon), Oncorhynchus mykiss (Rainbow trout) (Salmo gairdneri), Pollachius vixens (saithe), Salmo salar (Atlantic salmon), and Salmo trutta (Brown trout). Outbreaks of ISAV are responsible for large losses in salmon farms in several countries.

Sequence analyses of ISA virus genomes have been performed by several laboratories for over a decade and much information is available on the evolution of different strains of the virus. However, some ISAV strains apparently carry little or no lethal hemorrhagic disease, and it has not been possible to tell which sequence structures are related to lethality. This uncertainty makes the exclusion of lethal strains of ISAV difficult or impossible when deciding which salmon eggs to use to stock new or old salmon aqua farms. Salmon farms are an increasingly important source of food in many areas of the world. Uncertainty concerning the lethality of strains of ISAV is present in Atlantic salmon, in Canada, where ISAV is acknowledged to exist, but also has led to concern in the West coast of North America about whether lethal ISAV exists in Pacific Canadian salmon farms and whether it has reached farmed salmon in Alaska and non-farmed salmon in the open oceans.

Management of ISAV in aquaculture globally and locally has experienced a lack of effective vaccines and an absence of methods for determining if an isolate of ISAV represents a health threat to an aquaculture population where the threat is economically sufficient to warrant intervention. Identification of relative lethality in ISAV would provide a benchmark against which fish farmers could determine when intervention made economic sense and would provide a benchmark for policy makers to determine against which outbreaks certain public policy actions should be undertaken. Identification of relative lethality in ISAV likewise provides makers of vaccines and other therapeutics with targets for control to provide fish farmers with an economically-relevant vaccine. When researchers, commercial growers, and government officials have advanced knowledge of the presence and lethality of a strain of ISAV, they have crucial additional time for preparations of vaccines and other prophylactic measures in advance of a spreading outbreak.

There is a continuing need in the art for quantitative methods of differentiating, preventing, and treating ISAV infections and outbreaks. There is additionally a continuing need in the art for therapies against ISAV that apply across strains and across time.

Replikin peptides are a family of small peptides that have been correlated with the phenomenon of rapid replication in influenza, malaria, West Nile virus, foot and mouth disease, and many other pathogens. See, e.g., WO 2008/143717. Replikin peptides have likewise been generally correlated with the phenomenon of rapid replication in viruses, organisms, and malignancies.

Identification of Replikin peptides has provided targets for detection and treatment of pathogens, including vaccine development against virulent pathogens such as influenza virus, malaria, West Nile virus, and foot and mouth disease virus. See, e.g., WO 2008/143717. In general, knowledge of and identification of this family of peptides enables development of effective therapies and vaccines for any pathogen that harbors Replikins. The phenomenon of the association of Replikins with rapid replication and virulence has been fully described in U.S. Pat. No. 7,189,800; U.S. Pat. No. 7,176,275; U.S. Pat. No. 7,442,761; U.S. Pat. No. 7,894,999, U.S. Pat. No. 8,050,871, and U.S. application Ser. No. 12/108,458. Both Replikin concentration (number of Replikins per 100 amino acids) and Replikin composition have been correlated with the functional phenomenon of rapid replication.

There is a continuing need for monitoring Replikin sequences in ISAV to identify compounds for therapies that target ISAV. There is also a need to develop Replikin-based therapies that are effective across strains and within strains as they mutate over time.

In response to these continuing needs and despite extensive efforts in the fisheries industry to understand lethality in ISAV and to track and predict outbreaks of ISAV, applicants have now surprisingly applied their previous discovery of Replikin chemistry in the virus genome structure to methods of identifying relatively lethal strains of ISAV and methods of predicting outbreaks of ISAV. They have likewise now surprisingly provided methods of identifying conserved targets in emerging strains of ISAV against which vaccines may be developed prior to or at the outset of an outbreak. Such vaccine development can be undertaken in as few as seven days.

### SUMMARY OF THE INVENTION

The present invention provides compounds for diagnostic, therapeutic, and/or preventive purposes against ISAV and methods of identifying relative levels of lethality resulting from infections and outbreaks in populations of strains of infectious salmon anemia virus (ISAV), including variant strains of ISAV.

A first non-limiting aspect of the present invention provides an isolated or synthesized protein fragment or peptide comprising at least one peptide sequence that is at least 50% homologous with at least one Replikin peptide sequence identified in an infectious salmon anemia virus. In a non-limiting embodiment, the at least one peptide sequence may be at least 80% homologous with at least one Replikin peptide sequence identified in an infectious salmon anemia virus. In another non-limiting embodiment, the isolated or synthesized protein fragment or peptide may comprise at least one Replikin peptide sequence identified in an infectious salmon anemia virus or at least one homologue of the at least one Replikin sequence identified in an infectious salmon anemia virus. The isolated or synthesized protein fragment or peptide may consist essentially of at least one Replikin peptide sequence identified in an infectious salmon anemia virus or at least one homologue of said at least one Replikin peptide sequence identified in an infectious salmon anemia virus. The isolated or synthesized protein fragment or peptide may consist of at least one Replikin peptide sequence identified in an infectious salmon anemia virus or at least one homologue of said at least one Replikin peptide sequence identified in an infectious salmon anemia virus. Another non-limiting embodiment provides an isolated or synthesized peptide sequence comprising at least one functional fragment of a Replikin sequence identified in ISAV.

In a non-limiting embodiment, the isolated or synthesized protein fragment or peptide may comprise at least one Replikin peptide sequence of SEQ ID NO(s): 1-18 or at least one homologue of SEQ ID NO(s): 1-18. In a non-limiting embodiment, the isolated or synthesized protein fragment or peptide may consists essentially of at least one Replikin peptide sequence of SEQ ID NO(s): 1-18 or at least one homologue of SEQ ID NO(s): 1-18. In another non-limiting embodiment, the isolated or synthesized protein fragment or peptide may consist of at least one Replikin peptide sequence of SEQ ID NO(s): 1-18 or at least one homologue of SEQ ID NO(s): 1-18. Another non-limiting embodiment provides an isolated or synthesized peptide sequence comprising at lest one functional fragment of at least one Replikin peptide sequence of SEQ ID NO(s): 1-18.

A non-limiting embodiment of the first aspect of the invention provides an isolated or synthesized protein, protein fragment, polypeptide, or peptide comprising at least one Replikin peptide of an ISAV virus. A further embodiment of the first aspect of the invention provides an isolated or synthesized protein, protein fragment, polypeptide, or peptide comprising at least one peptide sequence that is at least 30%, 40%, 50%, 60%, 70%, 80%, 90% 95%, or 100%, homologous with at least one Replikin peptide sequence identified in an ISAV virus. In a non-limiting embodiment, the at least one sequence is one of SEQ ID NO(s): 1-18.

In a further non-limiting embodiment of the first aspect of the present invention, the isolated or synthesized protein, protein fragment, polypeptide, or peptide consists of 7 to about 50 amino acids comprising at least one peptide A, wherein said peptide A is at least 30%, 40%, 50%, 60%, 70%, 80%, 90%, 95%, or 100%, homologous with at least one Replikin peptide sequence identified in an ISAV. In one non-limiting embodiment, said at least one Replikin peptide sequence identified in an ISAV is at least one peptide sequence of SEQ ID NO(s): 1-18.

In a further non-limiting embodiment of the first aspect of the present invention, the isolated or synthesized protein, protein fragment, polypeptide, or peptide consists essentially of a Replikin peptide identified in ISAV. In a further non-limiting embodiment, the Replikin peptide sequence identified in an ISAV is at least one peptide sequence of SEQ ID NO(s): 1-18. A further non-limiting embodiment provides a peptide consisting of any one of SEQ ID NO(s): 1-18.

Another non-limiting embodiment of the first aspect of the invention provides a biosynthetic composition comprising the protein, protein fragment, polypeptide, or peptide of an aspect of the invention. In a further non-limiting embodiment, the biosynthetic composition consists essentially of a Replikin peptide of an ISAV or consists of a Replikin peptide of an ISAV. In a non-limiting embodiment, an isolated protein, protein fragment, polypeptide, or peptide is chemically synthesized by solid phase methods.

A second non-limiting aspect of the present invention provides an immunogenic and/or blocking composition comprising at least one protein, protein fragment, polypeptide, or peptide of any one of the above-listed proteins, protein fragments, polypeptides, or peptides including and not limited to comprising at least one Replikin peptide sequence identified in an infectious salmon anemia virus or at least one homologue of said at least one Replikin peptide identified in an infectious salmon anemia virus or at least one functional fragment of at least one Replikin peptide sequence identified in ISAV. In a non-limiting embodiment of the second aspect of the present invention, the immunogenic and/or blocking compound comprises at least one peptide sequence of SEQ ID NO(s): 1-18. In a further non-limiting embodiment, the immunogenic and/or blocking composition comprises at least one peptide consisting essentially of any one of SEQ ID NO(s): 1-18. In further non-limiting embodiment, the immunogenic and/or blocking composition comprises at least one peptide consisting of any one of SEQ ID NO(s): 1-18 or at least one functional fragment of any one of SEQ ID NO(s): 1-18.

A third non-limiting aspect of the present invention provides a vaccine comprising at least one protein, protein fragment, polypeptide, or peptide of any one of the above-listed proteins, protein fragments, polypeptides, or peptides. In a non-limiting embodiment of the third aspect of the present invention, the vaccine comprises at least one peptide sequence of any one of SEQ ID NO(s): 1-18, comprises at least one peptide sequence consisting essentially of any one of SEQ ID NO(s): 1-18, and/or comprises at least one peptide sequence consisting of any one of SEQ ID NO(s): 1-18 or at least one functional fragment of any one of SEQ ID NO(s): 1-18 or at least one functional fragment of a Replikin peptide sequence identified in ISAV.

In a further non-limiting embodiment of the third aspect of the present invention, the vaccine comprises a mixture of a plurality of peptide sequences of any of SEQ ID NO(s): 1-18 and/or a mixture of a plurality of homologues of peptide sequences of any of SEQ ID NO(s): 1-18. In a further non-limiting embodiment, the vaccine comprises a mixture of a plurality of peptide sequences consisting essentially of any one or more of SEQ ID NO(s): 1-18. In a further non-limiting embodiment, the vaccine comprises a mixture of a plurality of peptide sequences consisting of any one or more of SEQ ID NO(s): 1-18. In a further non-limiting embodiment, the vaccine comprises a mixture of a plurality of peptides consisting of each of SEQ ID NO(s): 1-18.

In another non-limiting embodiment of the third aspect of the invention, the vaccine comprises a mixture of Replikin peptides. In a non-limiting embodiment, the vaccine comprises an approximately equal molar mixture of the isolated or synthesized peptides of SEQ ID NO(s): 1-18. In a further non-limiting embodiment, the vaccine comprises approximately equal weight of the isolated or synthesized peptides of SEQ ID NO(s): 1-18.

In a further non-limiting embodiment, the vaccine comprises a pharmaceutically-acceptable carrier and/or adjuvant. In a further non-limiting embodiment, the vaccine is for the treatment or prevention of ISAV infection. In a further non-limiting embodiment, the vaccine is directed against NBISA01, 485/9/97, 1490/98, 301/98, 810/9/99, 835/9/98, Bergen, U5575-1, 390/98, 485/9/97, 832/98, 912/99, Glesvaer/2/90, 7833-1, SK-05:90, SK-05:144, or any other strain of ISAV.

A fourth non-limiting aspect of the invention provides an antibody, antibody fragment, or binding agent that binds to at least a portion of an amino acid sequence of at least one protein, protein fragment, polypeptide, or peptide comprising a peptide sequence that is 30%, 40%, 50%, 60%, 70%, 80%, 90%, or 95% or more homologous with at least one Replikin peptide sequence identified in ISAV. In a further embodiment, the at least one Replikin peptide sequence identified in ISAV is at least one peptide sequence of SEQ ID NO(s): 1-18.

A fifth non-limiting aspect of the present invention provides a method of making a vaccine comprising: selecting at least one isolated or synthesized protein, protein fragment, polypeptide, or peptide comprising at least one peptide sequence that is at least 30%, 40%, 50%, 60%, 70%, 80%, 90% or 95%, or 100% homologous with at least one Replikin peptide sequence identified in ISAV as a component of a vaccine; and making said vaccine. In a non-limiting embodiment, the method of making a vaccine comprises selecting at least one isolated or synthesized peptide of SEQ ID NO(s): 1-18, as at least one component and making said vaccine with the at least one component.

In another non-limiting embodiment, the method of making a vaccine comprises selecting at least two, three, four, five, six, seven, eight, nine, ten, eleven, twelve, thirteen, fourteen, fifteen, sixteen, seventeen, or eighteen or more isolated or synthesized Replikin peptide sequences identified in ISAV and/or isolated or synthesized functional fragments of Replikin peptide sequences identified in ISAV. In a further embodiment, the isolated or synthesized Replikin peptide sequences or functional fragments of Replikin peptide sequences identified in ISAV comprise at least one peptide sequence of SEQ ID NO(s): 1-18, at least one functional fragment of at least one peptide sequence of SEQ ID NO(s): 1-18, or at least one functional fragment of at least one Replikin peptide sequence identified in ISAV. In another non-limiting embodiment, the at least one isolated or synthesized protein, protein fragment, polypeptide, or peptide has the same amino acid sequence as at least one protein, protein fragment, polypeptide or peptide identified in a relatively lethal strain of ISAV up to seven days, one month, six months, one year, two years, or three years prior to making said vaccine.

A sixth non-limiting aspect of the present invention provides a method for preventing or treating ISAV infection comprising administering at least one isolated or synthesized protein, protein fragment, polypeptide, or peptide comprising at least one peptide sequence to a fish, where the peptide sequence is at least 30%, 40%, 50%, 60%, 70%, 80%, 90% or 95%, or 100%, homologous with at least one Replikin peptide identified in ISAV. In a further non-limiting embodiment, the Replikin peptide sequence is at least one peptide sequence of SEQ ID NO(s): 1-18. In a non-limiting embodiment, the at least one isolated or synthesized protein fragment, polypeptide, or peptide consists of at least one peptide sequence that is at least 30%, 40%, 50%, 60%, 70%, 80%, 90%, or 95% or more homologous with at least one of the peptide sequences of SEQ ID NO(s): 1-18. In another non-limiting embodiment, the at least one isolated or synthesized peptide of SEQ ID NO(s): 1-18 is administered to a fish. In a further non-limiting embodiment the at least one Replikin peptide sequence is at least one peptide sequence of SEQ ID NO(s): 1-18.

A seventh non-limiting aspect of the present invention provides a method of differentiating between relatively more lethal and relatively less lethal forms of infectious salmon anemia virus (ISAV). A first non-limiting embodiment provides a method of identifying and/or diagnosing a relatively more lethal form of infectious salmon anemia virus comprising determining the Replikin concentration of at least one portion of at least one protein of at least one isolate of ISAV or at least one portion of at least one gene that expresses at least one protein of the at least one isolate of ISAV and comparing the Replikin concentration of the at least one isolate of ISAV to a comparable Replikin concentration in at least one other isolate of ISAV. In a further non-limiting embodiment, the at least one portion of at least one protein comprises the entirety of at least one protein expressed in ISAV and the comparable Replikin concentration is the Replikin concentration of the entirety of the same protein expressed in ISAV from the at least one other isolate of ISAV. In a non-limiting embodiment, the Replikin concentration of the at least one isolate of ISAV is a mean of Replikin concentrations determined in a plurality of isolates of ISAV. In a further non-limiting embodiment, the Replikin concentration of the at least one other isolate of ISAV is a mean of Replikin concentrations determined in a plurality of other isolates of ISAV. In a further non-limiting embodiment, the plurality of isolates of ISAV is a collection of isolates isolated in a given year and the plurality of other isolates of ISAV is a collection of isolates isolated in a different year. In a further non-limiting embodiment, the Replikin concentration of the more lethal isolate of ISAV is 3.0 or greater, 4.0 or greater, or 5.0 or greater per 100 amino acid residues. In a further non-limiting embodiment, the Replikin concentration of the more lethal isolate of ISAV is 4.0 or greater per 100 amino acid residues. In a further non-limiting embodiment, the Replikin concentration of the more lethal isolate of ISAV is 4.6 per 100 amino acid residues or greater. In a further non-limiting embodiment, a vaccine is manufactured following the differentiation between relatively more lethal and relatively less lethal forms of ISAV. In a further non-limiting embodiment, the vaccine comprises at least one structure of the isolate of ISAV differentiated as relatively more lethal. In a further non-limiting embodiment, the vaccine comprises at least one Replikin peptide sequence identified in the isolate of ISAV differentiated as relatively more lethal.

In a further non-limiting embodiment of the seventh aspect of the present invention, the at least one portion of at least one gene expressing at least one protein is at least one portion of the pB1 region of the polymerase gene. In a further non-limiting embodiment, the at least one portion of at least one gene expressing at least one protein is the polymerase gene.

In a further non-limiting embodiment of the seventh aspect of the present invention, the Replikin concentration of the at least one isolate of ISAV is greater than the Replikin concentration of the at least one other isolate of ISAV. In a further non-limiting embodiment the Replikin concentration is a mean Replikin concentration of a plurality of isolates with standard deviation from the mean and the standard deviation from the mean is greater than the standard deviation from the mean Replikin concentration of a plurality of other isolates.

Another non-limiting embodiment of the seventh aspect of the invention provides a method of determining an increased probability of an outbreak of ISAV within about one year following an increase in Replikin concentration in an isolate of ISAV comprising identifying an increase in the concentration of Replikin sequences in at least one first isolate of ISAV as compared to at least one other isolate of ISAV wherein said at least one first isolate is isolated at a later time period than said one other isolate and wherein said increase in the concentration of Replikin sequences signifies the increased probability of the outbreak of ISAV within about one year following the increase in the concentration of Replikin sequences.

In a non-limiting embodiment, a method of prediction comprises: (1) obtaining a plurality of isolates of ISAV wherein at least one of said isolates is isolated about six months to about 3 years later than at least one other of said isolates; (2) analyzing the amino acid sequence of at least one protein or protein fragment in each isolate of the plurality of isolates for the presence and concentration of Replikin sequences; (3) comparing the concentrations of Replikin sequences in the at least one protein or protein fragment in each isolate of the plurality of isolates one to another; (4) identifying an increase in the concentration of Replikin sequences in said plurality of isolates over at least one time period of about six months or greater; and (5) predicting an outbreak of ISAV within about one month to about three years following said identified increase in the concentration of Replikin sequences. In another embodiment of the invention, the outbreak of ISAV is predicted within about six months. In a further embodiment of the invention, the outbreak of ISAV is predicted within about one year to about three years.

In a further non-limiting embodiment of the seventh aspect of the invention, the method of prediction further comprises comparison of the standard deviation from the mean of Replikin concentrations of isolates of ISAV from a given time period, such as a given month, a given year, or any other given time period. In a further non-limiting embodiment, the Replikin concentration is a mean Replikin concentration of a plurality of isolates with standard deviation from the mean and the standard deviation from the mean is greater than the standard deviation from the mean Replikin concentration of a plurality of other isolates.

### BRIEF DESCRIPTION OF THE DRAWINGS

FIG. 1 illustrates lethal Replikin concentrations in infectious salmon anemia virus (ISAV) in Norway from 1997 through 2012. The mean Replikin concentration in the pB1 gene area of isolates of ISAV from years in which isolates are available at the PubMed website of the National Center for Biotechnology Information (http://www.ncbi.nlm.nih.gov/) is illustrated by grey columns. Standard deviation from the mean among the population sample in a given year is illustrated by black standard error bars above the grey columns.

FIG. 2 illustrates lethal Replikin concentrations in infectious salmon anemia virus (ISAV) in Scotland from 1998 through 2011. The mean Replikin concentration in the pB1 gene area of isolates of ISAV from years in which isolates are available at the PubMed website of the National Center for Biotechnology Information (http://www.ncbi.nlm.nih.gov/) is illustrated by grey columns. Standard deviation from the mean among the population sample in a given year is illustrated by black standard error bars above the grey columns. A major outbreak of ISAV occurred in Scotland in 1999. FIG. 2 shows a marked increase in both the mean and the standard deviation of the mean for Replikin concentration in 1999. Increased Replikin concentration and increased standard deviation from the mean Replikin concentration in 1999 correlate with this Scottish outbreak.

FIG. 3 illustrates lethal Replikin concentrations in infectious salmon anemia virus (ISAV) in Chile from 2007 through 2010. The mean Replikin concentration in the pB1 gene area of isolates of ISAV from years in which isolates are available at the PubMed website of the National Center for Biotechnology Information (http://www.ncbi.nlm.nih.gov/) is illustrated by grey columns. Standard deviation from the mean among the population sample in a given year is illustrated by black standard error bars above the grey columns.

FIG. 4 illustrates lethal Replikin concentrations in infectious salmon anemia virus (ISAV) in Canada from 1997 through 2011. The mean Replikin concentration in the pB1 gene area of isolates of ISAV from years in which isolates are available at the PubMed website of the National Center for Biotechnology Information (http://www.ncbi.nlm.nih.gov/) is illustrated by grey columns. Standard deviation from the mean among the population sample in a given year is illustrated by black standard error bars above the grey columns.

FIG. 5 illustrates the accession number ADF36496 as identified from the PubMed website of the National Center for Biotechnology Information (http://www.ncbi.nlm.nih.gov/). The accession number discloses the amino acid sequence of the pB1 polymerase protein of an isolate of infectious salmon anemia virus (ISAV) isolated from an Atlantic salmon associated with a high mortality outbreak in Chile in 2010. The protein sequence comprises at least the following conserved Replikin peptide sequences: HWKAAKYIK (SEQ ID NO: 1); KEAVNRGHWK (SEQ ID NO: 2); HKYNERLK (SEQ ID NO: 3); KGYIWKHK (SEQ ID NO: 4); KRMWDIGNKH (SEQ ID NO: 5); KLIDEVEVIKKKKH (SEQ ID NO: 6); and HFRCMQGKQEVKGYIWK (SEQ ID NO: 7). The protein sequence disclosed in ADF36496 and portions of the protein sequence comprising Replikin sequences are useful for therapeutic purposes against highly lethal ISAV, for diagnostic purposes against highly lethal ISAV, and for purposes of identifying highly lethal isolates of ISAV.

FIG. 6 illustrates the accession number AAF72700 as identified from the PubMed website of the National Center for Biotechnology Information (http://www.ncbi.nlm.nih.gov/). The accession number discloses an amino acid sequence in the pB1 area of the polymerase protein of an isolate of infectious salmon anemia virus (ISAV) isolated from a host in the Bay of Fundy, Canada. The protein sequence comprises at least the following conserved Replikin peptide sequences (with various sequences identified as conserved in isolates from Canada, Norway, and Chile): HWKAAKYIK (SEQ ID NO: 1); KEAVNRGHWK (SEQ ID NO: 2); HKYNERLK (SEQ ID NO: 3); and KGYIWKHK (SEQ ID NO: 4). The protein sequence disclosed in AAF72700 and portions of the protein sequence comprising Replikin sequences are useful for therapeutic purposes against highly lethal ISAV, for diagnostic purposes against highly lethal ISAV, and for purposes of identifying highly lethal isolates of ISAV.

### DETAILED DESCRIPTION OF THE INVENTION

### Definitions

A “protein fragment” as used in this specification is any fragment of an expressed whole protein, which is any portion of an expressed whole protein where a “portion” of a protein is less than an expressed whole protein. A protein fragment reflects an expressed whole protein with one or more amino acids removed from the amino acid sequence of the expressed whole protein. A protein fragment may also reflect an amino acid sequence that is at least 30%, 40%, 50%, 60%, 70%, 80%, 90%, 95%, or 100% homologous with any portion of an expressed whole protein. A “polypeptide,” as used in this specification, is any portion of a protein fragment and is less than an expressed whole protein.

A “whole protein” or an “expressed whole protein” as used in this specification reflect a protein that is expressible from an intact gene of ISAV from a start codon to a stop codon. A whole protein or expressed whole protein may also reflect a whole protein or expressed whole protein that has been subject to cellular processing to create a protein that is capable of functioning within the virus replication system in a proper manner for virus replication. A protein fragment, polypeptide, or peptide “partially matches” the amino acid sequence of an expressed whole protein when the protein fragment, polypeptide, or peptide shares substantial homology with the expressed whole protein but at least one of the amino acids of the expressed whole protein are not present in the protein fragment, polypeptide, or peptide.

A “functional fragment” of a Replikin sequence as described herein is a fragment, variant, analog, or chemical derivative of a Replikin sequence that retains at least a portion of the immunological cross reactivity with an antibody specific for the Replikin sequence. A fragment of the Replikin sequence refers to any subset of the molecule. Variant peptides of the sequence may be made by direct chemical synthesis, for example, using methods well known in the art. An analog of a Replikin sequence to a non-natural protein or polypeptide is substantially similar to either the Replikin sequence of the protein or a fragment thereof. Chemical derivatives of a Replikin sequence contain additional chemical moieties.

As used herein, the term “preferentially binds” or “specifically binds” and related terms referencing the interaction of a binding molecule such as, for example, an antibody, and the structure to which it binds (antigen) means that the binding molecule preferentially recognizes the structure to which it binds even when present among other molecules (such as in a mixture of molecules). Specific or preferential binding of a binding molecule to a binding structure or an immunogenic portion of a binding structure is specific and preferential when the binding molecule binds to the structure or portion thereof and does not bind with the same level of affinity to other structures. Binding affinity may be determined by one of ordinary skill in the art using, for example, BIACORE, enzyme-linked immunosorbent assays, or radioimmuno assays. A binding molecule may cross-react with related antigens and preferably does not cross-react with affinity to unrelated antigens. Binding between a binding molecule and the structure to which it binds may be mediated by covalent or non-covalent attachment, or both.

As used herein a “vaccine” is any substance, compound, composition, mixture, or other therapeutic substance that, when administered to a human or animal via any method of administration known to the skilled artisan now or hereafter, produces an immune response, an antibody response, or a protective effect in the human or animal.

As used herein, a “Replikin sequence” is an amino acid sequence of 7 to 50 amino acid residues comprising (1) a first lysine residue located six to ten residues from a second lysine residue; (2) at least one histidine residue; and (3) at least 6% lysine residues, where the sequence is the shortest sequence comprising the first and second lysine residues of element (1) and the at least one histidine of element (3). A Replikin sequence may comprise more than two lysine residues and more than one histidine residue so long as at least two of the lysine residues and at least one histidine residue reflect the requirements of the definition of a Replikin sequence. For diagnostic, therapeutic, and preventive purposes, a Replikin sequence may or may not be the shortest sequence comprising the first and second lysine residues of element (1) and the at least one histidine residue of element (3).

The term “Replikin sequence” can also refer to a nucleic acid sequence encoding an amino acid sequence having 7 to about 50 amino acids comprising:
(1) at least one lysine residue located six to ten amino acid residues from a second lysine residue;
(2) at least one histidine residue; and
(3) at least 6% lysine residues.

As used herein, the term “peptide” or “protein” refers to a compound of two or more amino acids in which the carboxyl group of one amino acid is attached to an amino group of another amino acid via a peptide bond.

As used herein, an “isolated” peptide may be synthesized by organic chemical methods. An isolated peptide may also be synthesized by biosynthetic methods. An isolated peptide may also refer to a peptide that is, after purification, substantially free of cellular material or other contaminating proteins or peptides from the cell or tissue source from which the peptide is derived, or substantially free from chemical precursors or other chemicals when chemically synthesized by any method, or substantially free from contaminating peptides when synthesized by recombinant gene techniques or a protein or peptide that has been isolated in silico from nucleic acid or amino acid sequences that are available through public or private databases or sequence collections. An isolated peptide may be synthesized by biosynthetic or organic chemical methods.

Proteins, protein fragments, polypeptides, or peptides in this specification may be chemically synthesized by any method known to one of skill in the art now and hereafter. For example, isolated proteins, protein fragments, polypeptides, or peptides may be synthesized by solid phase synthesis. The production of these materials by chemical synthesis avoids the inclusion of (or the need to remove by purification) materials that are byproducts of other production methods such as recombinant expression or isolation from biological material. Such byproducts may include, for example, avian proteins associated with vaccines produced using birds' eggs, bacterial proteins associated with recombinant production in bacteria, or proteins or contaminants associated with any recombinant activity such as with productions of proteins or other sequences in insect cells.

An “encoded” or “expressed” protein, protein sequence, protein fragment sequence, or peptide sequence is a sequence encoded by a nucleic acid sequence that encodes the amino acids of the protein or peptide sequence with any codon known to one of ordinary skill in the art now or hereafter. It should be noted that it is well known in the art that, due to redundancy in the genetic code, individual nucleotides can be readily exchanged in a codon and still result in an identical amino acid sequence. As will be understood by one of ordinary skill in the art, a method of identifying a Replikin amino acid sequence also encompasses a method of identifying a nucleic acid sequence that encodes a Replikin amino acid sequence wherein the Replikin amino acid sequence is encoded by the identified nucleic acid sequence.

“Homologous” or “homology” or “sequence identity” as used in this specification indicate that an amino acid sequence or nucleic acid sequence exhibits substantial structural equivalence with another sequence, namely any Replikin peptide sequence (including SEQ ID NO(s): 1-18) identified in an isolate of ISAV or any nucleotide sequence encoding a Replikin peptide sequence in an isolate of ISAV (a redundancy in a coding sequence may be considered identical to a sequence encoding the same amino acid). To determine the percent identity or percent homology of an identified sequence, a sequence is aligned for optimal comparison purposes with any one of possible basis sequences. For purposes of this paragraph, a basis sequence is a Replikin sequence identified in an isolate of ISAV. Where gaps are necessary to provide optimal alignment, gaps may be introduced in the identified sequence or in the basis sequence. When a position in the identified sequence is occupied by the same amino acid residue or same nucleotide as the corresponding position in the basis sequence, the molecules are considered identical at that position (as used herein amino acid or nucleic acid “identity” is equivalent to amino acid or nucleic acid “homology”). To determine percent homology, the amino acid residues or nucleotides at corresponding amino acid positions or nucleotide positions are compared between the identified sequence and the basis sequence. The total number of amino acid residues or nucleotides in the identified sequence that are identical with amino acid residues or nucleotides in the basis sequence is divided by the total number of residues or nucleotides in the basis sequence (if the number of residues or nucleotides in the basis sequence is greater than the total number of residues or nucleotides in the identified sequence) or by the total number of amino acid residues or nucleotides in the identified sequence (if the number of residues or nucleotides in the identified sequence is greater than the total number of residues or nucleotides in the basis sequence). The final number is determined as a percentage. As such, the percent identity between the two sequences is a function of the number of identical positions shared by the sequences, taking into account the number of gaps (where a gap must be introduced for optimal alignment of the two sequences) and the length of each gap. Any structural or functional differences between sequences having sequence identity or homology will not affect the ability of the sequence to function as indicated in the desired application.

For example, SEQ ID NO: 19 (HAQDILEKEHNGKLCSLKGVRPLILK) is considered more than 86% homologous with the following sequence HAQDILEKEHNGKLCSLKGVRPXₙ₋₄LILK (SEQ ID NO: 20). The more than 86% homology between SEQ ID NO: 19 and SEQ ID NO: 20 is determined as follows: SEQ ID NO: 20 is the identified sequence. SEQ ID NO: 19 is the basis sequence. Upon alignment, SEQ ID NO: 20 is identical to SEQ ID NO: 19 in all 26 residues of SEQ ID NO: 19 (with a gap introduced for the four residues represented by Xₙ₌₄). To determine percent homology, then, the 26 aligned identical residues are divided by the total number of residues in SEQ ID NO: 20, namely 30 residues, giving 0.867 or more than 86% homology. SEQ ID NO: 19 is more than 86% homologous with HAQDXILEKEHNGKLCXSLKGVRXXPLILK (SEQ ID NO: 21) because it is identical to SEQ ID NO: 21 in all residues except for the residues represented by the four X residues.

In a further example, SEQ ID NO: 22 (KEHNGKLCSLKGVRPLILK) is more than 68% homologous with KEHNGKLCSLKGK (SEQ ID NO: 23). SEQ ID NO: 22 is the basis sequence and has 19 residues. SEQ ID NO: 23 is the reference sequence and has 13 residues that are identical to SEQ ID NO: 23 but VRPLIL (residues 20-25 of SEQ ID NO: 19) is not present between the glycine at position 12 and the terminal lysine at position 13 (all of the other residues are identical). To determine percent homology, then, the 13 aligned identical residues are divided by the total number of residues in SEQ ID NO: 22, namely 19 residues, giving 0.684 or more than 68% homology.

To determine homology between an identified sequence that is contained in a larger polypeptide, protein fragment, or protein, and a basis sequence, the polypeptide, protein fragment, or protein must first be optimally aligned with the basis sequence. Upon alignment of the sequences, the residue in the identified sequence that is farthest to the amino-terminus of the polypeptide, protein fragment, or protein and identical to a residue in the basis sequence that is farthest to the amino-terminus of the basis sequence is considered the amino-terminal residue of the identified sequence. Likewise, upon alignment, the residue in the identified sequence that is farthest to the carboxy-terminus of the polypeptide, protein fragment, or protein and identical to a residue in the basis sequence that is farthest to the carboxy-terminus of the basis sequence is considered the carboxy-terminal residue of the identified sequence.

Concerning gaps, the number of gaps in either the basis sequence or the identified sequence should be limited to the number of gaps allowable without significantly compromising the function of the identified sequence as compared to the basis sequence. In general, many gaps in the sequence of the basis peptide or in the sequence of the identified peptide are allowed based on homology as defined herein. Relatively more gaps are allowed if the lysines and histidines that create the definition of the Replikin peptide are identically shared between the basis peptide and the identified peptide. Relatively more gaps are also allowed if the lysines and histidines that create the definition of the Replikin peptide are shared at least in close position (for example within ten, nine, eight, seven, six, five, four, three, two, or one amino acid residue). If some of the lysine residues and histidine residues that create the definition of the Replikin peptide are not present in the identified peptide, fewer gaps may be allowed. Nevertheless, if the identified peptide functions similarly to the basis peptide, any number of gaps is allowed. In general, three or more gaps are allowed in the sequence of the basis peptide or in the sequence of the identified peptide within ten amino acid residues of the basis peptide if no lysines or histidines are present in the identified peptide. Two or more gaps or one or more gaps are also allowed. Nevertheless, if the identified sequence provides the same or a similar function to the basis sequence, more gaps are allowed up to the number of gaps that will provide a homology of 30%, 40%, 50%, 60%, 70%, 80%, 90%, 95%, or more homology. Additionally, where the lysines and histidines of the Replikin definition are present in both the identified peptide and the basis peptide, there should be no limit on how many gaps are allowed.

The presence of lysines and histidines providing for the Replikin definition in an identified peptide requires significantly less homology because the lysines and the histidines of the Replikin definition provide for conservation of Replikin function. For example, in Table 8 and the description thereof in columns 62 and 63 in U.S. Pat. No. 7,442,761, a highly mutable tat protein in HIV is described and analyzed. As may be seen from Table 8 in U.S. Pat. No. 7,442,761, in tat protein of HIV, which is essential for replication in the virus, lysines and histidines that are essential to maintaining the Replikin definition within a key Replikin peptide in the protein are observed to be 100% conserved, while substitutions in amino acid residues that are not essential to maintaining the Replikin definition are commonly substituted. The conservation of the key amino acids for maintaining the Replikin definition is understood to provide a specific survival function for HIV. The same phenomenon is seen in influenza. See U.S. Pat. No. 7,442,761, column 62, lines 42-45.

As used herein, “Replikin Count” or “Replikin concentration” refers to the number of Replikin sequences per 100 amino acids in a protein, protein fragment, virus, or organism. A higher Replikin concentration in a first strain of a virus or organism has been found to correlate with more rapid replication of the first virus or organism as compared to a second, earlier-arising or later-arising strain of the virus or organism having a lower Replikin concentration. Replikin concentration is determined by counting the number of Replikin sequences in a given sequence, wherein a Replikin sequence is a peptide of 7 to 50 amino acid residues comprising (1) a first lysine residue six to ten residues from a second lysine residue, (2) at least one histidine residue, (3) and 6% or more lysine residues where the Replikin sequence is the shortest sequence comprising the first and second lysine residues of element (1) and the at least one histidine residue of element (2). A Replikin sequence may comprise more than two lysine residues and more than one histidine residue so long as there is at least one lysine residue six to ten residues from a second lysine residue and at least one histidine residue. A Replikin sequence for the purpose of determining Replikin concentration as described in this paragraph may also be a nucleic acid that encodes a Replikin peptide sequence defined according to this paragraph.

### Methods of Identifying Relative Lethality in Isolates of ISAV

One non-limiting aspect of the present invention provides methods of differentiating relative lethality among isolates and/or outbreaks of ISAV in fish. Compounds for diagnostic, therapeutic, and/or preventive purposes in ISAV and therapies for the prevention and treatment of ISAV are provided based on the disclosed methods of differentiation.

A non-limiting embodiment of the invention provides a method of identifying a relatively more lethal form of infectious salmon anemia virus comprising determining the Replikin concentration of at least one portion of at least one protein of at least one isolate of ISAV or at least one portion of at least one gene that expresses at least one protein of the at least one isolate of ISAV and comparing the Replikin concentration of the at least one isolate of ISAV to a comparable Replikin concentration in at least one other isolate of ISAV. In a further non-limiting embodiment, the at least one portion of at least one protein comprises the entirety of the at least one protein expressed in ISAV and the comparable Replikin concentration is the Replikin concentration of the entirety of the same protein expressed in ISAV from the at least one other isolate of ISAV. For purposes of comparison, the Replikin concentration of one protein of a first isolated ISAV may be compared to the Replikin concentration of a fragment of the same protein of a second isolate so long as the fragment of the second isolate is sufficiently long to include the portions of the first isolate containing Replikin sequences.

In a non-limiting embodiment, the Replikin concentration of the at least one isolate of ISAV is a mean of Replikin concentrations determined in a plurality of isolates of ISAV. In a further non-limiting embodiment, the Replikin concentration of the at least one other isolate of ISAV is a mean of Replikin concentrations determined in a plurality of other isolates of ISAV. For comparison purposes, a mean Replikin concentration may reflect sequences of whole proteins and sequences of fragments of proteins. In a further non-limiting embodiment, the plurality of isolates of ISAV is a collection of isolates isolated in a given year and the plurality of other isolates of ISAV is a collection of isolates isolated in a different year.

In a further non-limiting embodiment, the at least one portion of at least one gene expressing at least one protein is at least one portion of the pB1 region of the polymerase gene. In a further non-limiting embodiment, the at least one portion of the at least one gene expressing at least one protein is the pB1 region of the polymerase gene. In a further non-limiting embodiment, the at least one portion of at least one gene expressing at least one protein is the polymerase gene.

In a further non-limiting embodiment, the Replikin concentration of the at least one isolate of ISAV is greater than the Replikin concentration of the at least one other isolate of ISAV. In a further non-limiting embodiment the Replikin concentration is a mean Replikin concentration of a plurality of isolates with standard deviation from the mean and the standard deviation from the mean is greater than the standard deviation from the mean Replikin concentration of a plurality of other isolates.

### Methods of Predicting Outbreaks of ISAV

One non-limiting aspect of the present invention provides a method of determining an increased probability of an outbreak of ISAV within about six months to about three years following an increase in Replikin concentration in an isolate of ISAV comprising identifying an increase in the concentration of Replikin sequences in at least one first isolate of ISAV as compared to at least one other isolate of ISAV wherein said at least one first isolate is isolated later than said at least one other isolate is isolated, and wherein said increase in the concentration of Replikin sequences signifies the increased probability of the outbreak of ISAV within about six months to about three years following the increase in the concentration of Replikin sequences. In a non-limiting embodiment, the first isolate of ISAV is isolated at least about six months later than the at least one other isolate.

In a non-limiting embodiment, a method of prediction comprises: (1) obtaining a plurality of isolates of ISAV wherein at least one of said isolates is isolated later (less than six months later or about six months to about 3 years later) than at least one other of said isolates; (2) analyzing the amino acid sequence of at least one protein or protein fragment in each isolate of the plurality of isolates for the presence and concentration of Replikin sequences; (3) comparing the concentrations of Replikin sequences in the at least one protein or protein fragment in each isolate of the plurality of isolates one to another; (4) identifying an increase in the concentration of Replikin sequences in said plurality of isolates over at least one time period (said period may be one day, one week, one month, or six months or greater); and (5) predicting an outbreak of ISAV following said identified increase in the concentration of Replikin sequences. In one embodiment of the invention, the outbreak of ISAV is predicted within about six months to about five years. In a further embodiment of the invention, the outbreak of ISAV is predicted within about one year to about three years.

In a further non-limiting embodiment of the aspect of the invention, the method of prediction further comprises comparison of the standard deviation of the mean of Replikin concentrations of isolates of ISAV from a given time period, such as a given month, a given year, or any other given time period. In a further non-limiting embodiment the Replikin concentration is a mean Replikin concentration of a plurality of isolates with standard deviation from the mean and the standard deviation from the mean is greater than the standard deviation from the mean Replikin concentration of a plurality of other isolates.

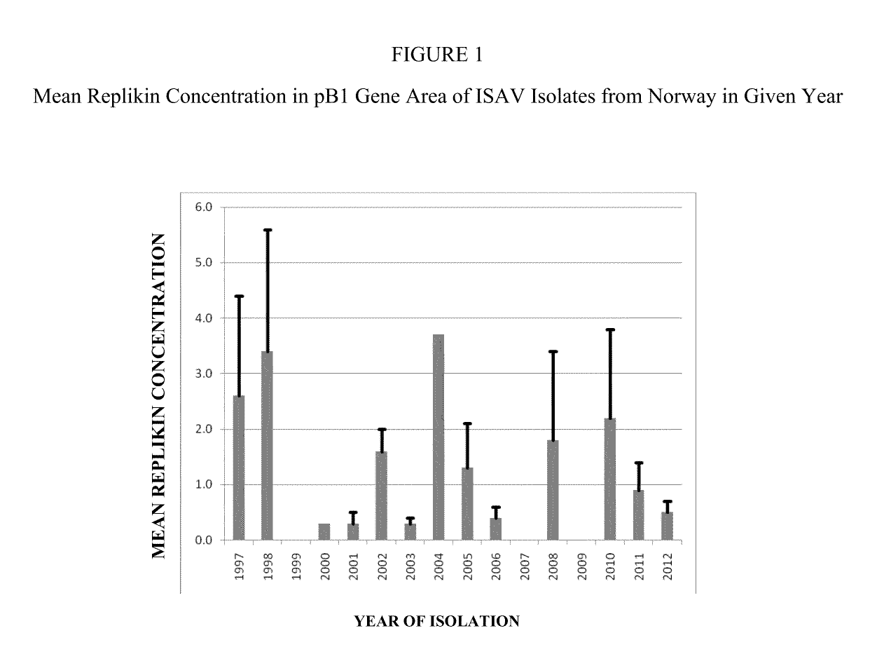

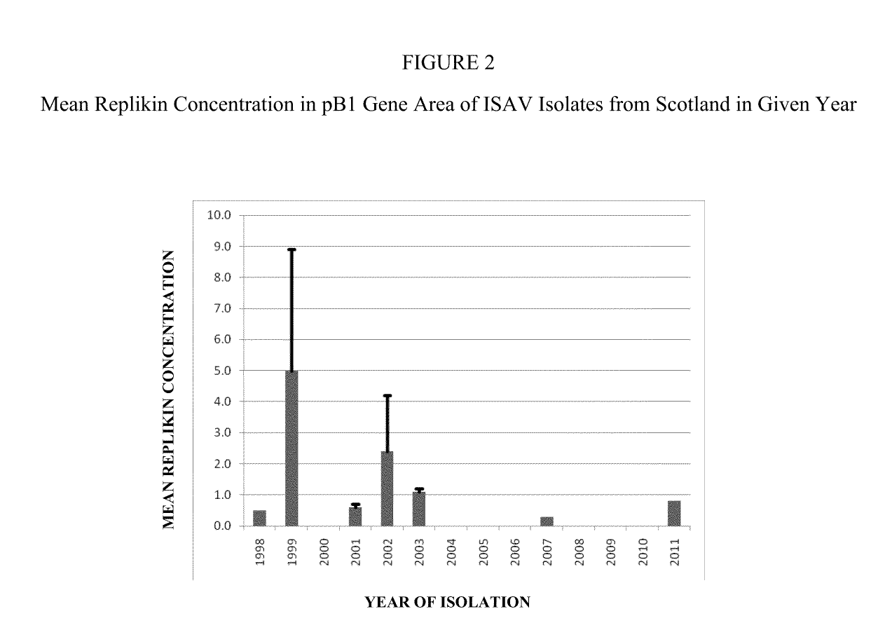

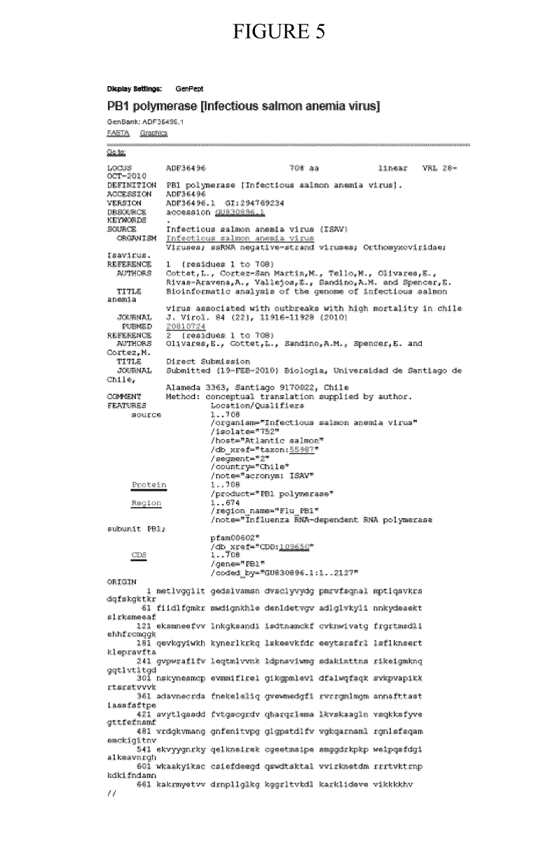

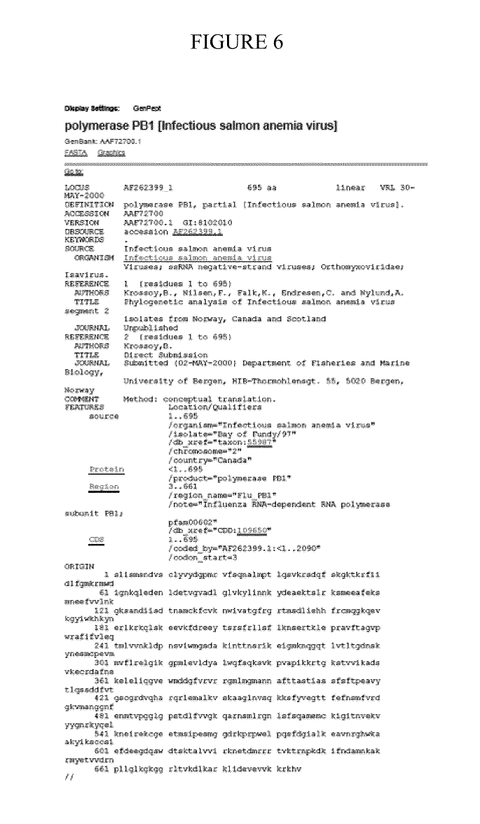

For example, FIG. 2 demonstrates a marked increase in both the mean and the standard deviation of the mean for Replikin concentration in 1999 in isolates of ISAV from Scotland. Both increased Replikin concentration and increased standard deviation from the mean Replikin concentration in 1999 correlate with an outbreak of ISAV in Scotland. This correlation has been seen in other pathogens including, for example, influenza, malaria, taura syndrome virus, white spot syndrome virus, foot and mouth disease, and other diseases. See, e.g., FIGS. 1-21 in WO 2008/143717.

### Lethal Replikin Concentrations Evolving in the Infectious Salmon Anemia Virus (ISAV) Genome in Canada Exceed Counts in Norway, Scotland, and Chile

Outbreaks of Infectious Salmon Anemia Virus (ISAV) are responsible for large losses in salmon farms in several countries. The peak Replikin concentrations of the pB1 regions, known to be related to lethality in influenza and other viruses, when examined in ISAV genomes, were found to be four times greater in Canadian salmon than in related ISAV in Scotland, Norway and Chile. This would suggest that the decline in salmon yields in recent years in Canadian and U.S. waters both farmed and in open oceans, may at least in part be due to ISAV. A completely synthetic Replikin vaccine has been formulated against ISAV.

Sequence analyses of the ISAV genomes have been performed by several laboratories for over a decade and much information is available on the evolution of different strains of the virus. However, some ISAV strains apparently carry little or no lethal hemorrhagic disease, and it has previously not been possible to tell which sequence structures are related to lethality. This uncertainty has made the exclusion of lethal strains of ISAV difficult or impossible when deciding which salmon eggs to use to stock new or old salmon aqua farms. These farms are an increasingly important source of food in many areas of the world. This uncertainty is present in Atlantic salmon, in Canada, where ISAV is acknowledged to exist, but also has led to concern in the West coast of North America about whether lethal ISAV exists in Pacific Canadian salmon farms and whether it has reached farmed salmon in Alaska and non-farmed salmon in the open oceans. Methods are provided herein for employing Replikin peptide sequences (a specific class of genomic peptides related to rapid replication) to allow the uncertainty now present in global fisheries to be resolved. Specific epitopes for the design and construction of synthetic vaccines against ISAV are also provided. Synthetic vaccines based on Replikin sequences have previously been shown to be effective in H5N1 influenza virus (see WO 2010/123519, Example 1) and taura syndrome virus (see WO 2008/156914, Examples 2 and 3) and vaccines have been designed against many other pathogens and cancers including, for example, SARS (see WO/2006/088962, Example 6), malaria (see WO 2003/05880, Example 4), and glioblastoma (see WO 2003/05880, Table 2). Nevertheless, no such vaccine has been designed for ISAV and until the disclosure provided herein, the industry had no expectation that such a vaccine could be designed.

Replikin sequence information has previously provided accurate prediction of lethal outbreaks in several strains of influenza, foot and mouth disease, and other viruses, as well as with E. Coli. One aspect of the present invention now provides methods of predicting lethal outbreaks in ISAV and the data provided herein affords the fisheries industry with a warning of outbreaks to come, with particular concern for the Canadian (and therefore North American) fishery.

In addition to Mean and Standard Deviation measures of the Replikin concentrations, the highest Replikin concentrations observed in a given year are also indications of the most extreme representatives in the population of ISAV at a given time, that is, the most mutated in favor of lethality, since the more increased the Replikin concentration, the higher the mortality (see extreme in glioblastoma multiforme brain cancer where the peak concentrations are 324 and compare to other cancers, see WO 2010/017514, FIG. 1).

As may be seen in FIG. 2, lethal Replikin concentrations in ISAV in Scotland from 1998 through 2011 have been observed. A major outbreak of ISAV occurred in Scotland in 1999. FIG. 2 shows a marked increase in both the mean and the Standard deviation of the mean for Replikin concentration in 1999. Increased Replikin concentration and increased standard deviation from the mean Replikin concentration in 1999 correlate with this Scottish outbreak. Such correlations have been observed in other outbreaks of other strains of other viruses (see, e.g., WO 2008/143717 FIGS. 1-8, 10-13, and 15-21) and other non-virus pathogens such as malaria (see, e.g., WO 2008/143717 FIG. 9) but the skilled artisan in the fisheries industry had no expectation that the same correlation would be observed in ISAV in salmon. In years subsequent to 1999, both Replikin concentration of ISAV isolates and lethality of ISAV infections (which measurements share a correlation) have declined. This decline may also be seen in the decline of the Replikin concentration in malaria, for example. See, e.g., WO 2008/143717 FIG. 9.

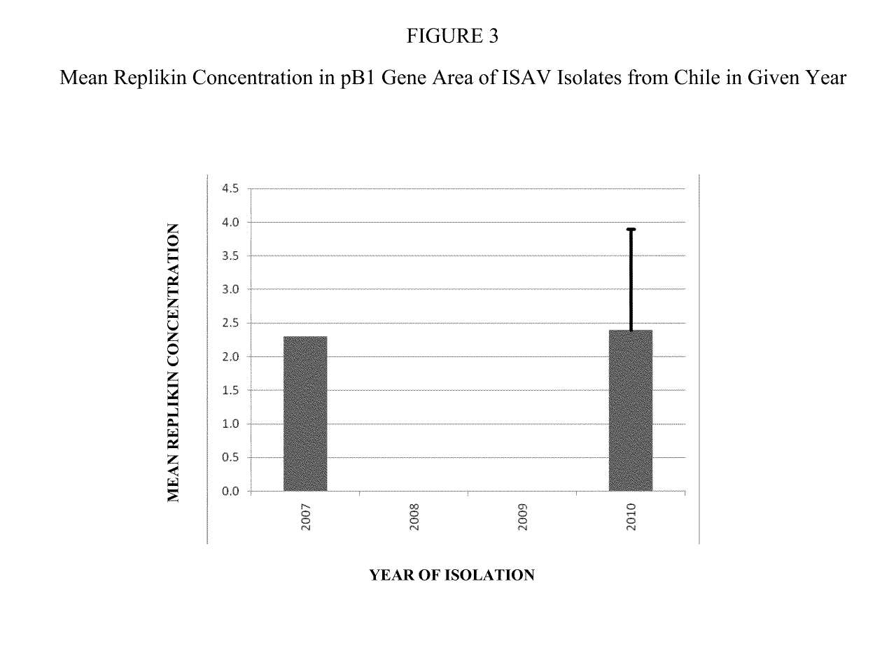

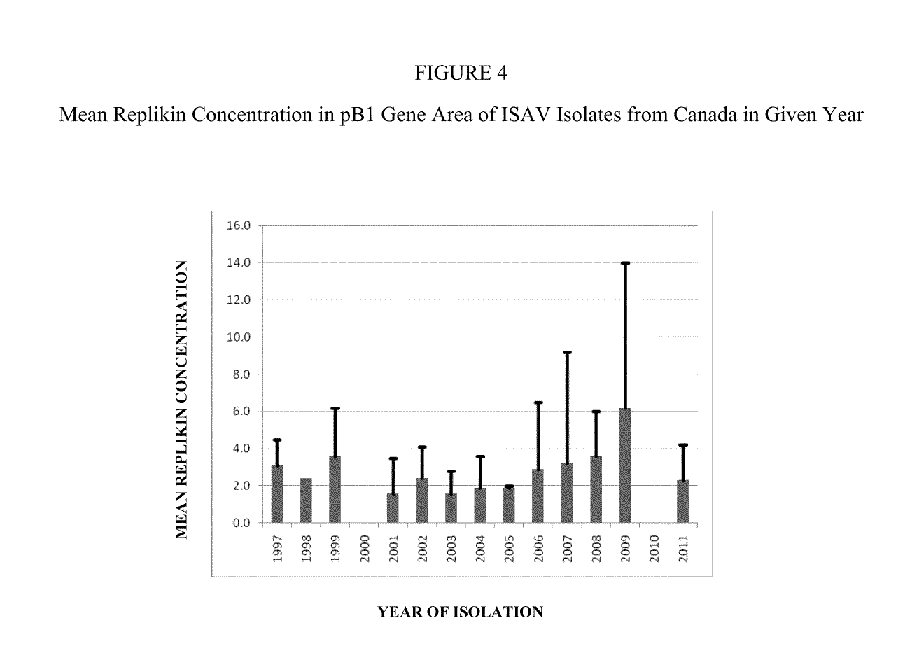

Over the past decade or so, salmon eggs were exported to Chile from Scotland and Norway. Apparently through contaminated eggs, ISAV was transmitted to Chile, with outbreaks from 2000 on. See FIG. 3. Salmon eggs were also transported to Canada from both Scotland and Norway. The presence of ISAV in salmon in Canada is seen in the FIG. 4 in the grey columns with black error bars providing standard deviation of the mean. As may be seen in FIGS. 1-4, the size of the standard deviation of the mean of Replikin concentration in a given time period (in these cases, in a given year) reflects the heterogeneity of the population with regard to mutated species of ISAV. Higher standard deviations reflect that some isolates in a population possess high Replikin concentrations and provide a warning that the portion of the population having high Replikin concentrations may expand.

Statistical analysis provides levels of certainty concerning differentiation of Replikin concentrations among isolates or groups of isolates of ISAV. P values indicate degree of statistical certainty with reference to the relationship between differences in Replikin concentration and relative lethality. A p value of less than 0.10 reflects less statistical certainty than a p value of less than 0.05, which reflects less statistical certainty than a p value of less than 0.01, which provides less statistical certainty than a p value of less than 0.001. Differentiation of Replikin concentrations may be made at p values of less than 0.1, 0.05, 0.01, and 0.001 or whatever statistical device applied by those of skill in the art. See, e.g., WO 2009/132209, FIGS. 3 and 4 and description thereof.

### Replikin Peptide Sequences Available for Therapies in ISAV Across Strains and in Different Countries

An aspect of the present invention provides compounds for diagnostic, therapeutic, and/or preventive purposes in ISAV, methods of differentiating relative lethality between one or more isolates of ISAV, and methods of designing therapies against ISAV based on compounds of the invention and differentiation of lethality among isolates of ISAV.

Compounds of one aspect of the invention comprise Replikin peptides and homologues of Replikin peptides identified in and isolated from different strains of ISAV and include Replikin peptides conserved over time in the same and different strains of ISAV. These Replikin peptides are useful when comprised in immunogenic compounds to provide a protective effect against ISAV infection including antagonism of the lethality of strains of ISAV. Replikin peptides that are conserved within strains of ISAV over time or across different strains of ISAV at conserved positions in the different strains of ISAV provide the ordinary skilled artisan with an expectation that the functionality of these peptides share commonality among various strains of ISAV and among various isolates of the same strain of ISAV at different times.

Eighteen peptides provided in an aspect of this invention were first identified as conserved in isolates of ISAV in different years from 1997 to 2011 and in different countries, including Canada, Norway, and Chile. The eighteen peptides are combined in a vaccine for administration to fish against challenge by ISAV. The vaccine is designed to generate an immune or blocking response in the fish that antagonizes infectivity, replication, and excretion of ISAV. Any virus that is not blocked on entry into the fish will be blocked intracellularly.

The eighteen peptides were surprisingly identified as conserved across countries and time. Each of the peptides was positioned in the pB1 gene area of the virus. The pB1 gene area of ISAV has been shown to be a Replikin Peak Gene area of the virus and Replikin Peak Gene areas of other viruses and pathogens have been demonstrated to be associated with lethality. See, e.g., WO 2008/143717, FIGS. 10-13, 16, 17, and 19. Based on the surprising data presented herein concerning the function of the pB1 gene area in lethality in ISAV (see FIGS. 1-4) as well as earlier data demonstrating the function of the pB1 gene area in lethality and based on the surprising commonality and conservation of homologues of pB1 gene area Replikin sequences over time and across countries in ISAV, any one or more of a pB1 sequence identified in ISAV is herein demonstrated to be useful as an immunogenic compound against any of the strains of ISAV in which a homologue of any of the sequences has been or will be identified. As a result, the applicants have developed methods of identifying other conserved Replikin peptides in ISAV as well as homologues of SEQ ID NO(s): 1-18 and homologues of any Replikin peptide sequence. These homologues are now available for use in immunogenic compounds that may be used against any strain of ISAV in which a homologue is identified. They are further available against strains of ISAV where the homologues are present in the pB1 gene area. In one aspect of the invention, a homologue may be 30%, 40%, 50%, 60%, 70%, 80%, 90%, 95%, or more or 100% homologous with a peptide against which the homologue is compared. The methods have provided peptides for a vaccine that may be applied for prevention or treatment of any strain of ISAV

Because the peptides disclosed in the vaccines herein described are peptides that are conserved over time in specific strains and shared between strains (also over time), one of skill in the art expects such peptides (and peptides that are similar in structure and function) to also be useful in immunogenic compounds for ISAV of various strains. This expectation is based on, for example, the function of the peptides identified herein and the commonality of structure and position of the peptides and their homologues as described herein as well as the functionality of the peptides and the homologues in the hemagglutinin protein area or hemagglutinin sequence equivalent protein area and pB1 gene area in different strains of ISAV. This expectation is also based in part on the conservation of Replikin peptides generally and the commonality of function of Replikin peptides across viruses and other organisms and across different viruses and organisms. See, e.g., Tables 7a, 8, 9, and 10 with descriptions and Examples 6 and 7 in U.S. application Ser. No. 11/355,120, filed Feb. 16, 2006 and Table 8 with description in U.S. Pat. No. 7,442,761, and FIGS. 1-18 WO2008/143717. For example, Replikin peptides have been shown to be broadly antigenic, to be conserved, and to be related to rapid replication and outbreaks across many different strains of influenza virus. See, e.g., WO 2008/143717. Additionally, the crucial lysine and histidine residues of Replikin peptides have been demonstrated to be related to rapid replication and to be conserved in fixed positions within functional proteins even in highly mutable viruses such as HIV. See, e.g., Table 8 with description in columns 62 and 63 in U.S. Pat. No. 7,442,761. Further, as described herein, the Replikin peptides and homologues disclosed herein are shown to be structurally and functionally related to lethality of ISAV.

As a result, the peptides and their homologues described herein are, among other things, antigenic, common to various strains of ISAV in both position and function, conserved in various strains of ISAV over time, conserved in specific positions in the hemagglutinin protein area and pB1 gene areas over time, conserved in their lysines and histidines within the Replikin structure, and associated with mechanisms of infectivity and/or lethality. One of ordinary skill in the art expects the Replikin peptides and their homologues described herein to be useful in immunogenic compounds for therapies against ISAV within strains, across strains, and across time.

An amino acid sequence of a protein fragment, polypeptide, or peptide is “derived from” an identified protein or gene area of ISAV (such as a pB1 gene area) if one of ordinary skill in the art would understand from the structure, history, or other relevant information of the amino acid sequence that it originated from an amino acid sequence of the identified protein or gene area of ISAV. Among other methods, one of ordinary skill may employ analysis of the homology of the amino acid sequence with the identified protein or gene area. One of ordinary skill may also employ the history of research used in developing the amino acid sequence to determine that the amino acid sequence is derived from an original sequence of the identified protein or gene area. One of ordinary skill would understand that a protein fragment, polypeptide, or peptide is derived from an identified protein, polypeptide, or peptide if it is traceable to the identified protein, polypeptide, or peptide, if it is deducible or inferable from the identified protein, polypeptide, or peptide, if the identified protein, polypeptide, or peptide is the source of the peptide, or if the protein fragment, polypeptide, or peptide is derived from the identified protein, polypeptide, or peptide as understood by one of skill in the art. One of ordinary skill may employ any method known now or hereafter for determining whether an amino acid sequence is derived from an identified protein or gene area of ISAV.

Shared and Conserved Replikin Peptide Sequences and their Homologues

Replikin sequences and their homologues provided by an aspect of the invention may be identified in strains of ISAV including any strain of ISAV known now or identified or known hereafter. Compounds of the invention may be conserved within strains of ISAV, across types within strains of ISAV, and across strains of ISAV. The compounds, because they are Replikin sequences, related to Replikin sequences, derived from Replikin sequences, identified as comprising Replikin sequences, or designed to comprise Replikin sequences, are related to rapid replication, virulence, and lethality in ISAV and comprise necessary structure for antigenicity. These characteristics of Replikin sequences have been previously established in other viruses and organisms (see, e.g., U.S. Pat. Nos. 7,894,999, 7,758,863 and WO 2008/143717) but have not previously been disclosed in ISAV; and the surprisingly effective utility of the Replikin sequence in predictions and therapies in ISAV is established herein. Compounds of the invention, including conserved Replikin peptides, are useful as immunogenic compounds to stimulate the immune system of a subject to produce an immune response, which may include production of antibodies or other binding molecules. Compounds of the invention are also useful in therapies such as vaccines. Compounds of the invention are likewise useful in producing antibodies, antibody fragments, or other binding or antagonizing agents, which may be used, among other things, for diagnostic and therapeutic purposes, including passive immunity.

The immunogenic compounds, antibodies (and other binding or antagonizing agents) and vaccines of the invention are useful against any strain of ISAV including, for example, NBISA01, 485/9/97, 1490/98, 301/98, 810/9/99, 835/9/98, Bergen, U5575-1, 390/98, 485/9/97, 832/98, 912/99, Glesvaer/2/90, 7833-1, SK-05:90, SK-05:144, or any other strain of ISAV. They are useful in any organism that is capable of producing an immune response or a protective effect. They are useful in fish. They are useful, for example, in Gadus morhua (Atlantic cod), Oncorhynchus kisutch (Coho salmon), Oncorhynchus mykiss (Rainbow trout) (Salmo gairdneri), Pollachius vixens (saithe), Salmo salar (Atlantic salmon), and Salmo trutta (Brown trout), among others. The compounds of the invention are also useful for diagnostic purposes, including identifying rapidly replicating, virulent, or lethal strains of virus.

The compounds of the invention may be conserved in strains of ISAV in various geographic regions including the strains listed in Table 4 isolated from Prince Edward Island. For example, the following eighteen peptides and their homologues as described herein are provided as an aspect of the invention as isolated or synthesized peptides, as immunogenic compounds, as vaccines, and as targets for antibodies and binding agents of the invention, among other things: HWKAAKYIK (SEQ ID NO: 1); KEAVNRGHWK (SEQ ID NO: 2); HKYNERLK (SEQ ID NO: 3); KGYIWKHK (SEQ ID NO: 4); KRMWDIGNKH (SEQ ID NO: 5); KLIDEVEVIKKKKH (SEQ ID NO: 6) HFRCMQGKQEVKGYIWK (SEQ ID NO: 7); KTVHWHLRVVK (SEQ ID NO: 8); KMTMMGKTVH (SEQ ID NO: 9); KMGDTRKEGYCH (SEQ ID NO: 10); KCWGMMFKTKSKMGDTRKEGYCH (SEQ ID NO: 11); HAIIFGKGEDKSGQNK (SEQ ID NO: 12); KVYGVLVDQLKLH (SEQ ID NO: 13); KLHGKDK (SEQ ID NO: 14); KLHGKDKVAGAKH (SEQ ID NO: 15); KDKVAGAKH (SEQ ID NO: 16); KQLHGQIHWK (SEQ ID NO: 17); and KFESPREFRKGH (SEQ ID NO: 18).

SEQ ID NO(s): 1-18 are sequences that were initially identified in ISAV as related to lethality in those strains of ISAV. Further investigation revealed identical sequences conserved in other isolates from different years and/or different countries. Conserved homologues would be expected to share the same functional characteristics in ISAV isolates where they are conserved. Conserved homologues are further identified in positions in the pB1 gene areas of various strains of ISAV where pB1 is associated with lethality.

Information on the conservation of homologous sequences across various strains of ISAV and in different regions provides sequences that offer immunogenic compounds for antagonism of all strains comprising these homologues across all regions having strains comprising these homologues. As a result, a vaccine is provided herein that offers cross-strain protection for a variety of strains of ISAV.

Replikin peptides in general are seen to be conserved across strains of ISAV. The key amino acid residues that provide for the Replikin sequence structure are the lysine and histidine residues wherein a Replikin sequence has at least one lysine on one terminus and at least one lysine or one histidine on the other terminus, at least one lysine that is six to ten residues from at least one other lysine, at least one histidine, and at least six percent lysines in total between the terminal lysine and the terminal lysine or histidine.

As may be seen in FIG. 10 of WO 2005/104754, when conserved homologous Replikin sequences are aligned one on top of the other over time, it is most apparent that fixed and conserved portions of the structure of Replikin sequences align in a series of posts or girders that illustrate, like the structure of a building, how key conserved amino acids provide constancy for the survival of a virus such as ISAV over time as it mutates to avoid immune recognition in its prospective host but maintains key functional genetic structures that provide for continued replication of the virus. These key functional genetic structures provide targets antagonized by Replikin-based therapies.

### Compounds and Compositions Comprising Peptides Homologous to ISAV Replikin Peptides

One aspect of the present invention provides a protein, a protein fragment, a polypeptide, or a peptide that comprises at least one peptide A homologous with at least one Replikin peptide identified in an isolate of ISAV. The Replikin peptide may be any Replikin peptide identified in an isolate of ISAV. The Replikin peptide may further be a Replikin peptide identified as conserved across strains or across regions in isolates of ISAV or any homologue of a Replikin peptide identified as conserved across strains or across regions in isolates of ISAV. For example, the Replikin peptide may be any one of SEQ ID NO(s): 1-18 or any homologue of any one of SEQ ID NO(s): 1-18.

Peptide A of the protein, protein fragment, polypeptide, or peptide may be 30%, 40%, 50%, 60%, 70%, 80%, 90%, or 95% or more homologous or 100% homologous with a Replikin peptide, including any of the peptides of SEQ ID NO(s): 1-18. A protein fragment or peptide may likewise be a peptide that consists of a peptide A that is homologous with a Replikin peptide of ISAV, including any of SEQ ID NO(s): 1-18. A peptide consisting essentially of or consisting of a Replikin peptide of ISAV, including any one of SEQ ID NO(s): 1-18, is also provided.

The amino acid sequence of the provided isolated or synthesized protein, protein fragment, polypeptide, or peptide may partially match an amino acid sequence of an expressed whole protein. At least one, five, ten, twenty, thirty, forty, fifty, one hundred, two hundred, three hundred, four hundred, five hundred, five hundred and fifty or more amino acid residues of the amino acid sequence of the expressed whole protein may not be present in the protein, protein fragment, polypeptide, or peptide. The amino acid sequence of an isolated or synthesized protein fragment, polypeptide, or peptide may also partially match the amino acid sequence of an expressed whole protein where at least one, ten, twenty, thirty, forty, fifty, sixty, seventy, eighty, ninety, one hundred, one hundred fifty, two hundred, two hundred fifty, three hundred, three hundred fifty, four hundred, four hundred fifty, five hundred, five hundred fifty or more amino acid residues of at least one terminus of the amino acid sequence of the expressed whole protein is (are) not present at least one terminus of said protein fragment, polypeptide, or peptide. Any additional number of amino acids may be situated on one or the other terminus or on both termini of the protein fragment, polypeptide, or peptide.

Because a Replikin peptide, such as SEQ ID NO(s): 1-18, is associated with rapid replication, infectivity, and/or lethality, in functional proteins in ISAV and because a Replikin peptide such as any one of SEQ ID NO(s): 1-18 are antigenic, inclusion of any Replikin peptide in a protein, protein fragment, polypeptide, or peptide does not negate the functional nature of the Replikin peptide. As such, antagonism of at least one Replikin peptide, including at least one of SEQ ID NO(s): 1-18 or a homologue of SEQ ID NO(s): 1-18 (with homology of 30% or greater) within a protein, protein fragment, polypeptide, or peptide would be expected to antagonize the replication, infectivity, and/or lethality of the protein fragment, polypeptide, or peptide.

A provided peptide may further be a peptide B of 7 to about 50 amino acid residues where peptide B contains a peptide A that is 30%, 40%, 50%, 60%, 70%, 80%, 90%, or 95% or more homologous or 100% homologous with any Replikin peptide, including one of SEQ ID NO(s): 1-18. A non-limiting peptide may further be a peptide A that is a Replikin peptide.

An isolated or synthesized protein, protein fragment, polypeptide, or peptide may consist of a peptide that is 30%, 40%, 50%, 60%, 70%, 80%, 90%, 95%, or 100% homologous with at least one of SEQ ID NO(s): 1-18 where the length of the peptide is no more than one, five, ten, twenty, thirty, forty, or fifty amino acid residues longer than a Replikin peptide of ISAV including the sequence of SEQ ID NO(s): 1-18 with which it is homologous. An isolated or synthesized protein fragment, polypeptide, or peptide may likewise be no more than one, two, three, four, five, six, seven, eight, nine, or ten amino acid residues longer than the Replikin sequence with which it is homologous (including, for example, SEQ ID NO(s): 1-18).

Another non-limiting embodiment provides a biosynthetic composition of the invention. The biosynthetic composition may comprise the isolated or synthesized protein, protein fragment, polypeptide, or peptide of an aspect of the invention disclosed herein. The biosynthetic composition may further consist essentially of a Replikin peptide of an ISAV. In a further embodiment, the Replikin peptide of an ISAV is at least one peptide having a sequence of SEQ ID NO(s): 1-18. A further non-limiting embodiment provides a biosynthetic composition consisting of a Replikin peptide of ISAV. In a non-limiting embodiment, the isolated protein fragment, polypeptide, or peptide of an aspect of the invention is chemically synthesized by solid phase methods.

An isolated or synthesized polypeptide or peptide may comprise a peptide A that has about the same number of amino acid residues as a peptide B, where peptide B is one of the peptides of SEQ ID NO: 1-18 and where the lysine residues and histidine residues in peptide A are conserved as compared to the lysine residues and histidine residues in peptide B. An isolated or synthesized polypeptide or peptide comprising peptide A may have up to 100 additional amino acid residues as compared to peptide B. Some or all of the up to 100 additional amino acid residues may be positioned toward the amino-terminus and/or carboxy-terminus of the lysine or histidine termini of peptide A. Some of the additional amino acid residues may be positioned within the lysine or histidine termini of peptide A so long as a level of homology is maintained between peptide A and peptide B that retains at least some of the functionality of the Replikin peptide of peptide B. Functionality may include, but is not limited to, antigenicity, rate of replication, antagonizability of a protein containing said peptide A or said peptide B, binding capacity of binding agents to peptides A or B, etc.

An isolated or synthesized polypeptide or peptide may also comprise up to about 90, about 80, about 70, about 60, about 50, about 40, about 30, about 20, about 10, about 5, about 4, about 3, about 2, or about 1 additional amino acid residues. The residues may be entirely outside of the Replikin structure or entirely within the Replikin structure or partially within and partially outside the Replikin structure. A level of homology should be maintained between peptides B and A when additional residues are present or are added. Residues outside of the Replikin structure are those residues on the amino-terminus or carboxy-terminus of the polypeptide or peptide as compared to the lysine or histidine termini of peptide A. Residues within the Replikin structure are those residues that are between the lysine or histidine termini of peptide A. An isolated or synthesized polypeptide or peptide may also consist of peptide A and peptide A may consist of peptide B.

An isolated or synthesized peptide may consist of a peptide of about 9 amino acid residues with a histidine residue within zero, one, two, three, four, or five residues of the amino-terminus of the peptide, wherein the histidine residue is considered to reside at position 1, and wherein relative to position 1 there is a lysine residue at position 3, a lysine residue at position 6, and a lysine residue at position 9, and wherein up to five additional residues may be present on the carboxy-terminus of the peptide after the lysine residue at position 9, and wherein up to one, two, three, four, or five additional residues may be present on the carboxy-terminus of the peptide after the lysine residue at position 9. If five residues are present on the amino-terminus of position 1 and five residues are present on the carboxy-terminus of position 9, the isolated or synthesized peptide will consist of about 19 amino acids. An example of an artificial ISAV homologue is XXXXXHXKXXKXXKXXXXX (SEQ ID NO: 24). Such an isolated or synthesized peptide is a homologue of SEQ ID NO: 1 and may be used as an immunogenic compound or as a component of a vaccine against any strain of ISAV.

An isolated or synthesized peptide may consist of about 10 amino acid residues with a lysine residue within zero, one, two, three, four, or five residues of the amino-terminus of the peptide, wherein the lysine residue is considered to reside at position 1, and wherein relative to position 1 there is a histidine residue at position 8, and a lysine residue at position 10, and wherein up to one, two, three, four, or five additional residues may be present on the carboxy-terminus of the peptide after the lysine residue at position 10. If five residues are present on each end of the peptide, it will consist of about 20 amino acids. An example of an artificial ISAV homologue is XXXXXKXXXXXXHXKXXXXX (SEQ ID NO: 25). Such an isolated or synthesized peptide is a homologue of SEQ ID NO: 2 and may be used as an immunogenic compound or as a component of a vaccine against any strain of ISAV.

An isolated or synthesized peptide may consist of about 8 amino acids residues with a histidine residue within zero, one, two, three, four, or five residues of the amino-terminus of the peptide, wherein the histidine residue is considered to reside at position 1, and wherein relative to position 1 there is a lysine residue at position 2 and a lysine residue at position 8, and wherein up to one, two, three, four, or five additional residues may be present on the carboxy-terminus of the peptide after the histidine residue at position 8. If five residues are present on each end of the peptide, it will consist of about 18 amino acids. An example of an artificial ISAV homologue is XXXXXHKXXXXXKXXXXX (SEQ ID NO: 26). Such an isolated or synthesized peptide is a homologue of SEQ ID NO: 3 and may be used as an immunogenic compound or as a component of a vaccine against any strain of ISAV.

An isolated or synthesized peptide may consist of about 8 amino acid residues with a lysine residue within zero, one, two, three, four, or five residues of the amino-terminus of the peptide, wherein the lysine residue is considered to reside at position 1, and wherein relative to position 1 there is a lysine residue at position 6, a histidine residue at position 7, and a lysine residue at position 8, and wherein up to one, two, three, four, or five additional residues may be present on the carboxy-terminus of the peptide after the lysine residue at position 8. If five residues are present on each end of the peptide, it will consist of about 18 amino acids. An example of an artificial ISAV homologue is XXXXXKXXXXKHKXXXXX (SEQ ID NO: 27). Such an isolated or synthesized peptide is a homologue of SEQ ID NO: 4 and may be used as an immunogenic compound or as a component of a vaccine against any strain of ISAV.

An isolated or synthesized peptide may consist of about 10 amino acid residues with a lysine residue within zero, one, two, three, four, or five residues of the amino-terminus of the peptide wherein, the lysine residue is considered to reside at position 1 and wherein relative to position 1 there is a lysine residue at position 9, a histidine residue at position 10, and wherein up to one, two, three, four, or five additional residues may be present on the carboxy-terminus of the peptide after the histidine residue at position 10. If five residues are present on each end of the peptide, it will consist of about 20 amino acids. An example of an artificial ISAV homologue is XXXXXKXXXXXXXKHXXXXX (SEQ ID NO: 28). Such an isolated or synthesized peptide is a homologue of SEQ ID NO: 5 and may be used as an immunogenic compound or as a component of a vaccine against any isolate of ISAV.

An isolated or synthesized peptide may consist of about 14 amino acid residues with a lysine residue within zero, one, two, three, four, or five residues of the amino-terminus of the peptide wherein, the lysine residue is considered to reside at position 1, and wherein relative to position 1 there is a lysine residue at position 10, a lysine residue at position 11, a lysine residue at position 12, a lysine residue at position 13, and a histidine residue at position 14, and wherein up to one, two, three, four, or five additional residues may be present on the carboxy-terminus of the peptide after the histidine residue at position 14. If five residues are present on each end of the peptide, it will consist of about 24 amino acids. An example of an artificial ISAV homologue is XXXXXKXXXXXXXXKKKKHXXXXX (SEQ ID NO: 29). Such an isolated or synthesized peptide is a homologue of SEQ ID NO: 6 and may be used as an immunogenic compound or as a component of a vaccine against any strain of ISAV.

An isolated or synthesized peptide may consist of about 17 amino acids with a histidine residue within zero, one, two, three, four, or five residues of the amino-terminus of the peptide wherein, the histidine residue is considered to reside at position 1, and wherein relative to position 1 there is a lysine residue at position 8, a lysine residue at position 12, and a lysine residue at position 17, and wherein up to one, two, three four, or five additional residues may be present on the carboxy-terminus of the peptide after the lysine residue at position 17. If five residues are present on each end of the peptide, it will consist of about 27 amino acids. An example of an artificial ISAV homologue is XXXXXHXXXXXXKXXXKXXXXKXXXXX (SEQ ID NO: 30). Such an isolated or synthesized peptide is a homologue of SEQ ID NO: 7 and may be used as an immunogenic compound or as a component of a vaccine against any strain of ISAV.

An isolated or synthesized peptide may consist of about 11 amino acid residues with a lysine residue within zero, one, two, three, four, or five residues of the amino-terminus of the peptide wherein, the lysine residue is considered to reside at position 1, and wherein relative to position 1 there is a histidine residue at position 4, a histidine residue at position 6, and a lysine residue at position 11, and wherein up to one, two, three, four, or five additional residues may be present on the carboxy-terminus of the peptide after the lysine residue at position 11. If five residues are present on each end of the peptide, it will consist of about 21 amino acids. An example of an artificial ISAV homologue is XXXXXKXXHXHXXXXKXXXXX (SEQ ID NO: 31). Such an isolated or synthesized peptide is a homologue of SEQ ID NO: 8 and may be used as an immunogenic compound or as a component of a vaccine against any strain of ISAV.

An isolated or synthesized peptide may consist of about 10 amino acid residues with a lysine residue within zero, one, two, three, four, or five residues of the amino-terminus of the peptide wherein, the lysine residue is considered to reside at position 1, and wherein relative to position 1 there is a lysine residue at position 7, and a histidine residue at position 10, and wherein up to one, two, three, four, or five additional residues may be present on the carboxy-terminus of the peptide after the histidine residue at position 10. If five residues are present on each end of the peptide, it will consist of about 20 amino acids. An example of an artificial ISAV homologue is XXXXXKXXXXXKXXHXXXXX (SEQ ID NO: 32). Such an isolated or synthesized peptide is a homologue of SEQ ID NO: 9 and may be used as an immunogenic compound or as a component of a vaccine against any strain of ISAV.

An isolated or synthesized peptide may consist of about 12 amino acid residues with a lysine residue within zero, one, two, three, four, or five residues of the amino-terminus of the peptide wherein, the lysine residue is considered to reside at position 1, and wherein relative to position 1 there is a lysine residue at position 7, and a histidine residue at position 12, and wherein up to one, two, three, four, or five additional residues may be present on the carboxy-terminus of the peptide after the histidine residue at position 12. If five residues are present on each end of the peptide, it will consist of about 22 amino acids. An example of an artificial ISAV homologue is XXXXXKXXXXXKXXXXHXXXXX (SEQ ID NO: 33). Such an isolated or synthesized peptide is a homologue of SEQ ID NO: 10 and may be used as an immunogenic compound or as a component of a vaccine against any strain of ISAV.

An isolated or synthesized peptide may consist of about 23 amino acid residues with a lysine residue within zero, one, two, three, four, or five residues of the amino-terminus of the peptide wherein, the lysine residue is considered to reside at position 1, and wherein relative to position 1 there is a lysine residue at position 8, a lysine residue at position 10, a lysine residue at position 12, a lysine residue at position 18, and a histidine residue at position 23, and wherein up to one, two, three, four, or five additional residues may be present on the carboxy-terminus of the peptide after the lysine residue at position 23. If five residues are present on each end of the peptide, it will consist of about 33 amino acids. An example of an artificial ISAV homologue is XXXXXKXXXXXXKXKXKXXXXXKXXXXHXXXXX (SEQ ID NO: 34). Such an isolated or synthesized peptide is a homologue of SEQ ID NO: 11 and may be used as an immunogenic compound or as a component of a vaccine against any strain of ISAV.

An isolated or synthesized peptide may consist of about 16 amino acid residues with a histidine residue within zero, one, two, three, four, or five residues of the amino terminus of the peptide wherein, the histidine residue is considered to reside at position 1, and wherein relative to position 1 there is a lysine residue at position 7, a lysine residue at position 11, and a lysine residue at position 16, and wherein up to one, two, three, four, or five additional residues may be present on the carboxy-terminus of the peptide after the lysine residue at position 16. If five residues are present on each end of the peptide, it will consist of about 26 amino acids. An example of an artificial ISAV homologue is XXXXXHXXXXXKXXXKXXXXKXXXXX (SEQ ID NO: 35). Such an isolated or synthesized peptide is a homologue of SEQ ID NO: 12 and may be used as an immunogenic compound or as a component of a vaccine against any strain of ISAV.

An isolated or synthesized peptide may consist of about 13 amino acid residues with a lysine residue within zero, one, two, three, four, or five residues of the amino terminus of the peptide, wherein the lysine residue is considered to reside at position 1, and wherein relative to position 1 there is a lysine residue at position 11 and a histidine residue at position 13 and wherein up to one, two, three, four, or five additional residues may be present on the carboxy-terminus of the peptide after the histidine residue at position 13. If five residues are present on each end of the peptide, it will consist of about 23 amino acids. An example of an artificial ISAV homologue is XXXXXKXXXXXXXXXKXHXXXXX (SEQ ID NO: 36). Such an isolated or synthesized peptide is a homologue of SEQ ID NO: 13 and may be used as an immunogenic compound or as a component of a vaccine against any strain of ISAV.

An isolated or synthesized peptide may consist of about 7 amino acid residues with a lysine residue within zero, one, two, three, four, or five residues of the amino terminus of the peptide, wherein the lysine residue is considered to reside at position 1, and wherein relative to position 1 there is a histidine residue at position 3, a lysine residue at position 5, and a lysine residue at position 7 and wherein up to one, two, three, four, or five additional residues may be present on the carboxy-terminus of the peptide after the lysine residue at position 7. If five residues are present on each end of the peptide, it will consist of about 17 amino acids. An example of an artificial ISAV homologue is XXXXXKXHXKXKXXXXX (SEQ ID NO: 37). Such an isolated or synthesized peptide is a homologue of SEQ ID NO: 14 and may be used as an immunogenic compound or as a component of a vaccine against any strain of ISAV.

An isolated or synthesized peptide may consist of about 13 amino acid residues with a lysine residue within zero, one, two, three, four, or five residues of the amino terminus of the peptide, wherein the lysine residue is considered to reside at position 1, and wherein relative to position 1 there is a histidine residue at position 3, a lysine residue at position 5, a lysine residue at position 7, a lysine residue at position 12, and a histidine residue at position 13 and wherein up to one, two, three, four, or five additional residues may be present on the carboxy-terminus of the peptide after the histidine residue at position 13. If five residues are present on each end of the peptide, it will consist of about 23 amino acids. An example of an artificial ISAV homologue is XXXXXKXHXKXKXXXXKHXXXXX (SEQ ID NO: 38). Such an isolated or synthesized peptide is a homologue of SEQ ID NO: 15 and may be used as an immunogenic compound or as a component of a vaccine against any strain of ISAV.

An isolated or synthesized peptide may consist of about 9 amino acid residues with a lysine residue within zero, one, two, three, four, or five residues of the amino terminus of the peptide, wherein the lysine residue is considered to reside at position 1, and wherein relative to position 1 there is a lysine residue at position 3, a lysine residue at position 8, and a histidine residue at position 9 and wherein up to one, two, three, four, or five additional residues may be present on the carboxy-terminus of the peptide after the histidine residue at position 9. If five residues are present on each end of the peptide, it will consist of about 19 amino acids. An example of an artificial ISAV homologue is XXXXXKXKXXXXKHXXXXX (SEQ ID NO: 39). Such an isolated or synthesized peptide is a homologue of SEQ ID NO: 16 and may be used as an immunogenic compound or as a component of a vaccine against any strain of ISAV.

An isolated or synthesized peptide may consist of about 10 amino acid residues with a lysine residue within zero, one, two, three, four, or five residues of the amino terminus of the peptide, wherein the lysine residue is considered to reside at position 1, and wherein relative to position 1 there is a histidine residue at position 4, a histidine residue at position 8, and a lysine residue at position 10 and wherein up to one, two, three, four, or five additional residues may be present on the carboxy-terminus of the peptide after the lysine residue at position 10. If five residues are present on each end of the peptide, it will consist of about 20 amino acids. An example of an artificial ISAV homologue is XXXXXKXXHXXXHXKXXXXX (SEQ ID NO: 40). Such an isolated or synthesized peptide is a homologue of SEQ ID NO: 17 and may be used as an immunogenic compound or as a component of a vaccine against any strain of ISAV.

An isolated or synthesized peptide may consist of about 12 amino acid residues with a lysine residue within zero, one, two, three, four, or five residues of the amino terminus of the peptide wherein, the lysine residue is considered to reside at position 1, and wherein relative to position 1 there is a lysine residue at position 10 and a histidine residue at position 12 and wherein up to one, two, three, four, or five additional residues may be present on the carboxy-terminus of the peptide after the histidine residue at position 12. If five residues are present on each end of the peptide, it will consist of about 22 amino acids. An example of an artificial ISAV homologue is XXXXXKXXXXXXXXKXHXXXXX (SEQ ID NO: 41). Such an isolated or synthesized peptide is a homologue of SEQ ID NO: 18 and may be used as an immunogenic compound or as a component of a vaccine against any strain of ISAV.

Any one of the above-listed isolated or synthesized peptides may have an amino-terminus at position 1 and a carboxy-terminus at the amino acid residue for which a position is expressly numbered where that expressly-numbered position is the farthest numbered position toward the carboxy-terminus of the peptide. For example, a homologue of SEQ ID NO: 7 (HFRCMQGKQEVKGYIWK) may have a terminal histidine at position 1 and a terminal lysine at position 17 or a homologue of SEQ ID NO: 4 (KGYIWKHK) may have a terminal lysine at position number 1 and a terminal lysine at position number 8.

The at least one isolated or synthesized protein, protein fragment, or peptide may also comprise at least one peptide A and at least one peptide C where peptide A is at least 30%, 40%, 50%, 60%, 70%, 80%, 90%, 95%, or 100% homologous with at least one Replikin peptide of ISAV, which may include a peptide of SEQ ID NO(s): 1-18, and where peptide C is at least 30%, 40%, 50%, 60%, 70%, 80%, 90%, 95%, or 100% homologous with at least one other Replikin peptide of ISAV, which also may include a peptide of SEQ ID NO(s): 1-18. Peptide C may be homologous with a different Replikin peptide than the peptide that peptide A is homologous with. The at least one isolated or synthesized protein, protein fragment, or peptide may comprise three or more peptides homologous with at least three different Replikin peptides.

All of the above-discussed proteins, protein fragments, polypeptides, and peptides comprise the functional unit of a homologue of a Replikin peptide present in or isolated from an ISAV. The Replikin peptide may be any one of SEQ ID NO(s): 1-18. Antagonism of any of the homologues of a Replikin peptide will antagonize replication in ISAV and if the Replikin peptide is present or isolated from the pB1 gene portion of ISAV, it will be expected to antagonize the lethality function in any strain of ISAV that share a homologue of any one of the sequences. As a result, the proteins, protein fragments, polypeptides, and peptides are useful as immunogenic compounds, therapeutic compounds, vaccines, and for other therapies directed to antagonizing the replication and/or lethality of a strain of ISAV. When comprised in a vaccine, disclosed proteins, protein fragments, polypeptides, and peptides are expected to be capable of limiting the excretion or shedding of ISAV such that the virus is limited in its spread from host to host or from host to reservoir to host, etc. As such, disclosed compounds are effective at limiting sources of ISAV infection. Likewise, any binding agent that binds one of the proteins, protein fragments, polypeptides, and peptides discussed above will antagonize the replication and/or lethality of a strain of ISAV and limit sources of ISAV infection such as transmission from host to host or from host to reservoir to host.

### Immunogenic Compositions Comprising Peptide Homologous to ISAV Replikin Peptides

A protein, protein fragment, or peptide comprising a Replikin peptide present or identified in an isolate of ISAV may be comprised in an immunogenic compound. The proteins, protein fragment, polypeptides, and peptides provided by an aspect of the invention comprise at least a portion that is homologous with a Replikin peptide or homologous with one of the ISAV Replikin peptides of SEQ ID NO(s): 1-18. These homologues are expected by one of ordinary skill in the art to stimulate the immune system of a subject upon sufficient exposure to produce antibodies against at least the homologous portion of the protein, protein fragment, polypeptide, or peptide and/or to produce a protective effect against ISAV. One of ordinary skill in the art would expect that antibodies or other binding agents arrayed against a protein or protein fragment comprising one of the antigenic homologues disclosed herein would be antagonistic to the protein or protein fragment.

One of ordinary skill would also expect an antagonist of one of these homologues to antagonize any ISAV that comprises a homologue within its pB1 gene area. Because homologues of SEQ ID NO(s): 1-18 have been shown to be conserved across strains and across regions in the pB1 gene area, one of ordinary skill would expect antagonism of such homologues to result in antagonism of ISAV replication. One of ordinary skill would further expect particular antagonism of the lethality mechanisms of ISAV when an immune system is stimulated against a homologue of any one or more of SEQ ID NO(s): 1-18.

As a result, one aspect of the present invention is a method of stimulating the immune system of a subject with at least one compound comprising at least one Replikin sequence identified in ISAV or at least one isolated or synthesized homologue of at least one Replikin sequence identified in ISAV. The at least one Replikin sequence of the compound reflects an immunogenic target against which the immune system of the subject responds. Because at least a functional portion of the immunogenic structure of the target is maintained in a functional fragment of the at least one Replikin sequence, a functional fragment of the Replikin sequence is likewise a target against which the immune system of the subject responds. The compound may comprise a protein comprising the at least one Replikin sequence or functional fragment thereof, a protein fragment, a polypeptide, or a peptide comprising the at least one Replikin sequence or functional fragment thereof. The compound may comprise more than one protein, protein fragment, polypeptide or peptide. The compound may further be a composition of a plurality of synthesized or isolated Replikin sequences.

### Vaccines Comprising Peptides Homologous to ISAV Replikin Peptides

An immunogenic compound provided as an aspect of the invention may be used as a component of a non-limiting vaccine against any strain of ISAV. A vaccine comprising one or more homologues of a Replikin peptide of ISAV may be used against ISAV. The vaccine may comprise one or more homologues of SEQ ID NO(s): 1-18. Likewise, a vaccine comprising one or more homologues of a Replikin peptide may be used against ISAV and may antagonize the replication and/or lethality of an ISAV infection. Further, mixtures of homologues of SEQ ID NO(s): 1-18 are provided as vaccines to antagonize the replication and/or lethality of an ISAV infection. Such vaccines are useful for antagonizing replication, lethality, and excretion or spread of ISAV.

One vaccine may comprise at least one protein, protein fragment, polypeptide, or peptide of any one or more of the proteins, protein fragments, polypeptides, or peptides of an aspect of the invention. The vaccine may further comprise at least one Replikin peptide of ISAV. One such Replikin peptide may be any one of SEQ ID NO(s): 1-18. A vaccine may comprise at least one peptide consisting essentially of any one of SEQ ID NO(s): 1-18 or at least one peptide consisting of any one of SEQ ID NO(s): 1-18. A vaccine may comprise a mixture of Replikin peptides of ISAV. The vaccine may comprise a plurality of peptides such as two, three, four, five, six, seven, eight, nine, ten, eleven, twelve, thirteen, fourteen, fifteen, sixteen, seventeen, or eighteen peptides of SEQ ID NO(s): 1-18. The peptide may consist essentially of any one or more SEQ ID NO(s): 1-18 or may consist of any one or more of SEQ ID NO(s): 1-18.

In another non-limiting embodiment, the vaccine comprises a mixture of peptides, wherein the mixture comprises isolated or synthesized peptides of SEQ ID NO(s): 1-18. In a non-limiting embodiment, the vaccine comprises an approximately equal molar mixture of the isolated or synthesized peptides of SEQ ID NO(s): 1-18. In a further non-limiting embodiment, the vaccine comprises approximately equal weight of the isolated or synthesized peptides of SEQ ID NO(s): 1-18.

The vaccine may comprise homologues of sequences of ISAV. SEQ ID NO(s): 15 and 16 are non-limiting examples of homologues. SEQ ID NO: 16 has the same amino acid residues as the residues at positions 5 through 13 of SEQ ID NO: 15. As a result, SEQ ID NO: 16 is 69% homologous with SEQ ID NO: 15. Likewise SEQ ID NO(s): 10 and 11 are non-limiting examples of homologues. SEQ ID NO: 10 has the same amino acid residues as the residues at positions 12 through 23 of SEQ ID NO: 11. As a result, SEQ ID NO: 10 is 52% homologous with SEQ ID NO: 11.

In a further non-limiting embodiment, the vaccine is for the treatment or prevention of ISAV infection. In a further non-limiting embodiment, the vaccine is directed against NBISA01, 485/9/97, 1490/98, 301/98, 810/9/99, 835/9/98, Bergen, U5575-1, 390/98, 485/9/97, 832/98, 912/99, Glesvaer/2/90, 7833-1, SK-05:90, SK-05:144, or any other strain of ISAV.

A vaccine may further comprise at least one Replikin peptide from the polymerase gene area of ISAV. The vaccine may further comprise at least one Replikin peptide from the pB1 gene area of the polymerase gene area of ISAV. The vaccine may further comprise at least one Replikin peptide from the hemagglutinin gene area of ISAV or from a hemagglutinin sequence equivalent gene area of ISAV. A vaccine may also comprise at least one Replikin peptide from the hemagglutinin gene area or a hemagglutinin sequence equivalent gene area and at least one Replikin peptide from the pB1 gene area. A vaccine may comprise a plurality of Replikin peptides from the pB1 gene area and a plurality of Replikin peptides from the hemagglutinin gene area or a hemagglutinin sequence equivalent gene area.

A vaccine may comprise a plurality of the shortest Replikin peptides from a polymerase gene area, a pB1 gene area, or a hemagglutinin gene area (or equivalent). A vaccine may comprise the shortest Replikin peptides from a pB1 gene area identified in an ISAV isolate or a plurality of ISAV isolates predicted to have a greater lethality than at least one other isolate of ISAV.

A vaccine may further comprise a plurality of the longest Replikin peptides from a polymerase gene area, a pB1 gene area, or a hemagglutinin gene area (or equivalent). A vaccine may comprise the longest Replikin peptides from a polymerase gene area, a pB1 gene area, or a hemagglutinin gene area (or equivalent) identified in an ISAV isolate or a plurality of ISAV isolates predicted to have a greater infectivity than at least one other isolate of ISAV and may comprise the longest Replikin peptides from a pB1 gene area identified in an ISAV isolate predicted to have a greater lethality than at least one other isolate of ISAV. A vaccine may also comprise a mixture of the shortest and longest Replikin peptides in the pB1 gene area.

A vaccine may be directed against any strain of ISAV including any strain listed in Table 4. Any of these vaccines may be synthesized in seven days or less, which allows for administration of vaccines that are a best fit for a particular virulent strain of virus.

A vaccine may be formulated with a pharmaceutically acceptable excipient, carrier, or adjuvant. One pharmaceutically acceptable carrier or excipient is water. Excipients, carriers, or adjuvants may include, but are not limited to, excipients, carriers and adjuvants known to those of skill in the art now or hereafter.

A composition of the invention may be formulated for delivery by any available route including, but not limited to parenteral (e.g., intravenous), intradermal, subcutaneous, oral, nasal, bronchial, ophthalmic, transdermal (topical), transmucosal or any other routes. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

Administration of the vaccine via any method may produce an immune response in a fish or other aquatic creature, it may further produce an antibody response in the fish or aquatic creature. In a further non-limiting embodiment, the vaccine may produce a protective effect in the fish or aquatic creature. For example, the vaccine of the present invention may be administered to Gadus morhua (Atlantic cod), Oncorhynchus kisutch (Coho salmon), Oncorhynchus mykiss (Rainbow trout) (Salmo gairdneri), Pollachius vixens (saithe), Salmo salar (Atlantic salmon), and Salmo trutta (Brown trout) or any other fish susceptible to infection from ISAV.

Generally, the dosage of peptides is in the range of from about 0.01 μg to about 500 mg, from about 0.05 μg to about 200 mg, about 0.075 μg to about 30 mg, about 0.09 μg to about 20 mg, about 0.1 μg to about 10 mg, about 10 μg to about 1 mg, and about 50 μg to about 500 μg. The skilled practitioner can readily determine the dosage and number of dosages needed to produce an effective immune response.

### Compositions Comprising any of SEQ ID NO(s): 1-18

A non-limiting composition is provided comprising one or more isolated or synthesized peptides that are 30%, 40%, 50%, 60%, 70%, 80%, 90%, 95% or more homologous with at least one of the peptides of SEQ ID NO(s): 1-18. A composition is provided comprising one or more isolated or synthesized peptides consisting essentially of or consisting of at least one peptide of SEQ ID NO(s): 1-18. A composition is further provided comprising two, three, four five, six, seven, eight, nine, ten, eleven, twelve, thirteen, fourteen, fifteen, sixteen, seventeen, or eighteen isolated or synthesized peptides of SEQ ID NO(s): 1-18.

A composition comprising a mixture of peptides is provided wherein the mixture comprises at least each of the isolated or synthesized peptides of SEQ ID NO(s): 1-18. A mixture is provided that is equimolar. A mixture is also provided that is equal by weight.

A composition of an aspect of the invention is a therapeutic composition. The therapeutic composition may provide, upon administration to a subject, a therapeutic effect against ISAV. The therapeutic composition may be administered to any animal susceptible to infection from ISAV. The therapeutic composition may be administered to a fish. A fish may include, but is not limited to, Gadus morhua (Atlantic cod), Oncorhynchus kisutch (Coho salmon), Oncorhynchus mykiss (Rainbow trout) (Salmo gairdneri), Pollachius vixens (saithe), Salmo salar (Atlantic salmon), Salmo trutta (Brown trout), any other salmon species, including Pacific salmon species, or any other fish susceptible to infection by ISAV.

### Conserved Replikin Peptides Across ISAV and Peptide Homologies

Identification of conserved Replikin peptides across strains of ISAV in different countries has provided for the development of vaccines that may be directed across strains of ISAV in different countries. Identification of conserved Replikin peptides in isolates of ISAV of any strain may be accomplished in any way known to one of skill in the art now or hereafter. One method is by review of in silico sequences provided at the PubMed website of the National Center for Biotechnology Information (http://www.ncbi.nlm.nih.gov/). Peptides that share exact identity or 100% homology with earlier identified Replikin peptides may be tracked using computer-searching methods. Peptides that share 30%, 40%, 50%, 60%, 70%, 80%, 90%, or 95% or more homology with an earlier identified Replikin peptide may also be tracked by computer methods.

For example, a vaccine has now been developed for prevention and treatment of infection from ISAV. See, e.g., Example 1 below. The sequences that are used in the vaccine in Example 1 have now been identified as conserved across countries and strains of ISAV. These sequences, homologues of these sequences, and proteins, protein fragments, polypeptides, and peptides comprising, consisting essentially of, or consisting of these sequences or their homologues are useful in identifying lethal strains of ISAV, treating infections from ISAV, and developing prophylactic therapies, such as vaccines, against infection from ISAV.

These proteins, fragment, polypeptides and peptides including any one of SEQ ID NO(s): 1-18 and/or homologues of any one of SEQ ID NO(s): 1-18 are expected by one of ordinary skill in the art to provide antigenicity that is comparable to any one of SEQ ID NO(s): 1-18. Further, because these homologues are often conserved in the pB1 gene areas of different strains of ISAV, these homologues are useful for developing antagonists against ISAV infections, including for vaccinating a subject with the homologous peptides to stimulate the immune system of the subject against the peptides and in-turn against ISAV proteins harboring such peptides or other homologues of such peptides.

Homology that is sufficient to produce a useful target for antagonism includes peptides that are 30%, 40%, 50%, 60%, 70%, 80%, 90%, 95%, or up to 100% homologous with any of SEQ ID NO(s): 1-18. Homology may be determined with peptides wherein gaps exists in the sequence that is being compared to any one of SEQ ID NO(s): 1-18 between amino acids that are identical to those of the peptide chosen from SEQ ID NO(s): 1-18.

Sequences that are conserved across strains of ISAV are excellent targets for controlling infectivity and lethality.

### Methods of Designing Vaccines

The invention also provides methods of designing and making vaccines. For example, the invention provides a method of making a vaccine comprising selecting at least one or more isolated or synthesized Replikin peptides present or identified in an isolate of ISAV. Such peptides may include any one or more of SEQ ID NO(s): 1-18 as a component of a vaccine and making said vaccine. The method may comprise selecting from 1 to up to 18 or more isolated or synthesized ISAV Replikin peptides as a component of a vaccine. The peptides may be identified in the pB1 gene area of the virus or may be identified in the hemagglutinin sequence equivalent gene area or may be identified in any area of the genome of ISAV. The method may comprise identifying one or more Replikin peptides in an emerging strain of ISAV up to about three years before the vaccine is made, up to about one year before the vaccine is made, up to about six months before the vaccine is made, or up to about seven days before the vaccine is made.

An emerging strain may be any strain of ISAV identified by one of skill in the art as predicted to expand in a population in hosts, or predicted to increase in virulence, morbidity, and/or mortality (lethality) in its hosts. An emerging strain may likewise be a strain of ISAV wherein Replikin concentration is observed to be increasing over time. An emerging strain may likewise be a strain of ISAV identified within a rising portion of Replikin cycle, following a peak in a Replikin cycle, following a step-wise rise in a Replikin cycle, or identified by a Replikin Count Virus Expansion Index as an emerging strain of virus. See WO 2009/132209, the contents of which are incorporated herein by reference.

A method of making a vaccine is also provided comprising: selecting at least one isolated or synthesized protein, protein fragment, polypeptide, or peptide comprising a homologue of a Replikin peptide (including, for example, SEQ ID NO(s): 1-18) as a component of a vaccine; and making said vaccine. An isolated or synthesized protein, protein fragment, polypeptide, or peptide may comprise a peptide that is 30%, 40%, 50%, 60%, 70%, 80%, 90% or 95%, or 100%, homologous with at least one Replikin peptide. At least two, three, four, five, six, seven, eight, nine, ten, eleven, twelve, or more homologues of Replikin peptides may be selected. Also, at least two, three, four, five, six, seven, eight, nine, ten, eleven, twelve, or more Replikin peptides may be selected. The isolated or synthesized protein, protein fragment, polypeptide, or peptide may have the same amino acid sequence as at least one protein, protein fragment, polypeptide or peptide identified in an emerging strain of ISAV up to one, two, or three or more years prior to making said vaccine. The at least one protein, protein fragment, polypeptide or peptide may be identified in an emerging strain of ISAV one week, one month, two months, three months, four months, five months, or six months prior to making said vaccine.

The invention also provides a kit for making a vaccine where the kit includes at least one isolated or synthesized Replikin peptide of ISAV (including, for example, at least one peptide of SEQ ID NO(s): 1-18 or homologue of SEQ ID NO(s): 1-18). The kit may also include two, three, four, and up to eighteen or more peptides of SEQ ID NO(s): 1-18 or homologues of SEQ ID NO(s): 1-18.

### Antibodies as Diagnostics and Therapies for Identified Replikin Sequences

In another aspect of the invention, isolated Replikin peptides may be used to generate antibodies, antibody fragments, or to generate or identify other binding agents, which may be used, for example, for diagnostic purposes or to provide passive immunity in an individual. See, e.g., U.S. application Ser. No. 11/355,120, filed Feb. 16, 2006 and U.S. application Ser. No. 12/010,027, filed Jan. 18, 2008 (each incorporated herein by reference in their entirety).

Various procedures known in the art may be used for the production of antibodies to Replikin sequences or to proteins, protein fragments, polypeptides, or peptides comprising Replikin sequences. Such antibodies include, but are not limited to, polyclonal, monoclonal, chimeric, humanized, single chain, Fab fragments and fragments produced by a Fab expression library. Antibodies that are linked to a cytotoxic agent or signaling moiety may also be generated. Antibodies may also be administered in combination with an antiviral agent. Furthermore, combinations of antibodies to different Replikins may be administered as an antibody cocktail.

For the production of antibodies, various host animals may be immunized by injection with a Replikin peptide or a combination of Replikin peptides, including, but not limited to, fish, rabbits, mice, rats, and larger mammals. Monoclonal antibodies to Replikins may be prepared using any technique that provides for the production of antibody molecules. These include but are not limited to the hybridoma technique originally described by Kohler and Milstein, (Nature, 1975, 256:495-497), the human B-cell hybridoma technique (Kosbor et al., 1983, Immunology Today, 4:72), and the EBV hybridoma technique (Cole et al., Monoclonal Antibodies and Cancer Therapy, Alan R. Liss, Inc., pp. 77-96). In addition, techniques developed for the production of chimeric antibodies (Morrison et al., 1984, Proc. Nat. Acad. Sci USA, 81:6851-6855) or other techniques may be used. Alternatively, techniques described for the production of single chain antibodies (U.S. Pat. No. 4,946,778) can be adapted to produce Replikin-specific single chain antibodies. Antibody fragments that contain binding sites for a Replikin may be generated by known techniques. For example, such fragments include but are not limited to F(ab′)2 fragments which can be produced by pepsin digestion of the antibody molecules and or fragments that can be generated by reducing the disulfide bridges of F(ab′)2 fragments. Alternatively, Fab expression libraries can be generated (Huse et al., 1989, Science, 246:1275-1281) to allow rapid and easy identification of monoclonal Fab fragments with the desired specificity.

Binding agents are provided including an antibody, antibody fragment, or binding agent that binds to at least a portion of an amino acid sequence of at least one protein, protein fragment, polypeptide, or peptide comprising at least one peptide A, where peptide A is at least 30%, 40%, 50%, 60%, 70%, 80%, 90% or 95%, or 100%, homologous with at least one Replikin peptide of ISAV, which may include, for example, at least one Replikin peptide of SEQ ID NO(s): 1-18. The amino acid sequence of a protein fragment, polypeptide, or peptide may partially match the amino acid sequence of an expressed whole protein where at least one, five, ten, twenty, thirty, forty, fifty, one hundred, two hundred, three hundred, four hundred, five hundred or more amino acid residues of the amino acid sequence of the expressed whole protein are not present in the protein fragment, polypeptide, or peptide. The amino acid sequence of the protein fragment, polypeptide, or peptide may also partially match the amino acid sequence of an expressed whole protein where at least one, ten, twenty, thirty, forty, fifty, sixty, seventy, eighty, ninety, one hundred, one hundred fifty, two hundred, two hundred fifty, three hundred, three hundred fifty, four hundred, four hundred fifty, five hundred, five hundred fifty or more amino acid residues of the amino acid sequence of at least one terminus of the expressed whole protein are not present at least one terminus of said protein fragment, polypeptide, or peptide.

Binding agents are also provided including an antibody, antibody fragment, or binding agent that binds to at least a portion of an amino acid sequence that is 30%, 40%, 50%, 60%, 70%, 80%, 90%, or 95% or more homologous with at least one Replikin peptide of ISAV. In a non-limiting embodiment, the length of peptide A may be no more than one, five, ten, twenty, thirty, forty, or fifty amino acid residues longer than the identified Replikin sequence with which it is homologous. Binding agents are also provided that bind to at least a portion of an amino acid sequence of at least one of SEQ ID NO(s): 1-18.

Binding agents may specifically or preferentially bind to the target protein, protein fragment, polypeptide, or peptide. Binding agents may specifically or preferentially bind to a homologue of at least one of SEQ ID NO(s): 1-18. Binding agents may likewise specifically or preferentially bind to a peptide consisting of any one of SEQ ID NO(s): 1-18. Binding agents may also specifically or preferentially bind to a portion of a peptide consisting of any one of SEQ ID NO(s): 1-18 including a single amino acid within a homologue of SEQ ID NO(s): 1-18, two amino acids, three amino acids, four amino acids, five amino acids, or any number of amino acids spread within or outside a homologue.

### Nucleic Acids and Compositions of Nucleic Acids

An isolated or synthesized nucleic acid sequence is also provided that encodes a protein, protein fragment, polypeptide, or peptide comprising at least one peptide A, where peptide A is at least 30%, 40%, 50%, 60%, 70%, 80%, 90% or 95%, or 100%, homologous with at least one Replikin peptide of ISAV. The at least one Replikin peptide may be any peptide of SEQ ID NO(s): 1-18. A nucleic acid sequence may also encode a protein, a protein fragment, a polypeptide, or a peptide where the amino acid sequence of the protein, protein fragment, polypeptide, or peptide partially matches the amino acid sequence of an expressed whole protein and at least one, two, three, four, five, ten, twenty, thirty, forty, fifty, one hundred, two hundred, three hundred, four hundred, five hundred or more amino acid residues of the amino acid sequence of the expressed whole protein are not present in the protein fragment, polypeptide, or peptide. Further, the amino acid sequence of the protein, protein fragment, polypeptide, or peptide may partially match the amino acid sequence of an expressed whole protein where at least one, two, three, four, five, ten, twenty, thirty, forty, fifty, sixty, seventy, eighty, ninety, one hundred, one hundred fifty, two hundred, two hundred fifty, three hundred, three hundred fifty, four hundred, four hundred fifty, five hundred, five hundred fifty or more amino acid residues of the amino acid sequence of at least one terminus of the expressed whole protein may not be present at least one terminus of the protein, protein fragment, polypeptide, or peptide

An isolated or synthesized nucleic acid sequence may also encode a peptide consisting of 7 to about 50 amino acid residues comprising at least one Replikin peptide, which may be one of the peptide sequences of SEQ ID NO(s): 1-18. It may also encode a peptide that is at least 30%, 40%, 50%, 60%, 70%, 80%, 90%, or 95% or more homologous with at least one of the peptide sequences of SEQ ID NO(s): 1-18. It may also encode a peptide consisting essentially of or consisting of at least one of the peptide sequences of SEQ ID NO(s): 1-18.

The invention further provides an immunogenic composition that comprises an isolated or synthesized nucleic acid provided above. The invention further provides a vaccine against ISAV comprising an isolated or synthesized nucleic acid provided above.

Anti-Sense Nucleic Acids and siRNA

The invention further provides a nucleic acid sequence that is antisense to a nucleic acid that encodes for any Replikin peptide present in or identified in an ISAV isolate. This may include one of SEQ ID NO(s): 1-18 or a small interfering nucleic acid sequence that interferes with a nucleic acid sequence that is 30%, 40%, 50%, 60%, 70%, 80%, 90%, or 95% or more homologous with a nucleic acid that encodes any Replikin peptide of ISAV including, for example, any one of SEQ ID NO(s): 1-18 or is 30%, 40%, 50%, 60%, 70%, 80%, 90%, 95% or more homologous with a nucleic acid that is antisense to a nucleic acid that encodes for any one of SEQ ID NO(s): 1-18.

The nucleotide sequence of the invention may be used in hybridization assays of biopsied tissue or blood, e.g., Southern or Northern analysis, including in situ hybridization assays, to diagnose the presence of a particular ISAV strain in a tissue sample or an environmental sample, for example. The present invention also provides kits containing antibodies specific for particular Replikins that are present in a particular isolate of ISAV, or containing nucleic acid molecules (sense or antisense) that hybridize specifically to a particular Replikin, and optionally, various buffers and/or reagents needed for diagnosis.

Also within the scope of the invention are oligoribonucleotide sequences that include antisense RNA and DNA molecules and ribozymes that function to inhibit the translation of Replikin-containing mRNA. Both antisense RNA and DNA molecules and ribozymes may be prepared by any method known in the art. The antisense molecules can be incorporated into a wide variety of vectors for delivery to a subject. The skilled practitioner can readily determine the best route of delivery, although generally intravenous or intramuscular delivery is routine. The dosage amount is also readily ascertainable.

An aspect of the invention further provides antisense nucleic acid molecules that are complementary to a nucleic acid of the invention, wherein the antisense nucleic acid molecule is complementary to a nucleotide sequence encoding a peptide of the invention. In particular the nucleic acid sequence may be anti-sense to a nucleic acid sequence that has been demonstrated to be conserved over a period of six months to one or more years and/or which are present in a strain of ISAV shown to have an increase in concentration of Replikin sequences relative to Replikin concentration in other ISAV strains.

An aspect of the invention also provides compositions comprising RNAi-inducing entities used to inhibit or reduce ISAV infection or replication including small interfering RNA, which is a class of about 10 to about 50 and often about 20 to about 25 nucleotide-long double-stranded RNA molecules. siRNA is involved in the RNA interference pathway, where it interferes with the expression of a specific gene such as the hemagglutinin gene or the pB1 gene area of ISAV. siRNAs also act in RNAi-related pathways, e.g., as an antiviral mechanism.

An effective amount of an RNAi-inducing entity is delivered to a cell or organism prior to, simultaneously with, or after exposure to ISAV. A dosage may be sufficient to reduce or delay one or more symptoms of ISAV infection. Compositions of the invention may comprise a single siRNA species targeted to a target transcript or may comprise a plurality of different siRNA species targeting one or more target transcripts.

The invention provides a small interfering nucleic acid sequence that is about 10 to about 50 nucleic acids in length and is 30%, 40%, 50%, 60%, 70%, 80%, 90%, or 95% or more homologous with a nucleic acid that encodes for any portion of an ISAV Replikin peptide including, for example, any portion of SEQ ID NO(s): 1-18 or is 30%, 40%, 50%, 60%, 70%, 80%, 90%, or 95% or more homologous with a nucleic acid that is antisense to a nucleic acid that encodes for any portion of a Replikin peptide, including, for example, a portion of one of SEQ ID NO(s): 1-18. In a further non-limiting embodiment, the nucleic acid sequence is about 15 to about 30 nucleic acids. In a further non-limiting embodiment, the nucleic acid sequence is about 20 to about 25 nucleic acids. In a further non-limiting embodiment, the nucleic acid sequence is about 21 nucleic acids.

### Advance Replikin-Based Information on Pathogenic Outbreaks Provides for Rapid Production of Vaccines

Advance information concerning Replikin peptides in expanding strains of pathogen allows for the rapid production of specific effective synthetic vaccines using one, or a combination, of Replikin peptides. Such synthetic vaccines have been demonstrated in rabbits, chickens, and shrimp. See, e.g., Example 1 herein, Examples 6 and 7 of U.S. application Ser. No. 11/355,120, filed Feb. 16, 2006, and Example 2 of U.S. application Ser. No. 12/108,458, filed Apr. 23, 2008. For example, a mixture of Replikin peptides administered orally to shrimp provided up to a 91% protective effect for shrimp challenged with taura syndrome virus. Taura syndrome virus is an often-lethal rapidly replicating pathogen that has a significant negative impact on the shrimp industry.

Synthetic Replikin vaccines have also been demonstrated in the H5N1 strain of influenza virus in chickens. For example, in a test of chickens administered a mixture of twelve H5N1 Replikin peptides from the hemagglutinin and pB1 gene areas intranasally, intraocularly, and by spray inhalation and challenged with low pathogenic H5N1 influenza isolated from a black duck in the state of North Carolina in the United States, a protective effect was observed at both the entry site of influenza (diminished antibody production in the serum was observed as compared to a control) and at excretion sites of influenza (influenza virus was not observed excreted in feces or saliva from treated chickens as compared to a control). See Example 1 below.

Administration of Replikin peptides in both shrimp and chickens appears to have provided a notable measure of mucosal immunity. For example, in Example 2 of U.S. application Ser. No. 12/108,458, a mixture of Replikin peptides was administered by mouth to shrimp later challenged with taura syndrome virus. The 91% protective effect of the vaccine is expected to have been a result, at least in part, of a mucosal immune-like response in the gut of the shrimp.

Likewise, in chickens, the administration of a mixture of Replikin peptides provided a protective effect against entry of the H5N1 virus. For example, as may be seen in Example 1 of U.S. application Ser. No. 12/581,112, filed Oct. 16, 2009 (incorporated herein by reference), three of six vaccinated chickens, when inoculated with H5N1 virus, produced no measurable amount of antibodies against H5N1 in their serum. Instead, the virus was apparently blocked by mucosal immunity from even entering the chickens' system. Some virus apparently entered the system of the chickens but was then blocked intracellularly. While the applicants do not wish to be bound by theory, the virus may have been blocked in its intracellular transport to the RNA or in synthesis of virus on RNA or in transport from the RNA to excretion. Wherever the block occurs, the fact is that the examination of the excreta of the chicken showed complete absence of virus. For those three chickens in which a serum immune response was measured (that is, virus did enter their system), the vaccine additionally provided a protective effect against replication of the virus in the chickens' system (no virus was excreted in the feces or saliva of the chickens). As such, mucosal immunity, in addition to other immunities, is an important aspect of the immunity imparted by Replikin-based vaccines.

### Example 1

### Synthetic Replikin Vaccine Against ISAV in Fish

A synthetic Replikin vaccine containing an approximately equal-parts-by-weight mixture of twelve ISAV Replikin peptides was designed for use against relatively lethal isolates of ISAV. The vaccine was engineered from sequences identified in the pB1 gene area of ISAV and confirmed to be conserved across regions (countries) and across time (conserved in ISAV strains from 1997 to 2011). Conservation was particularly noted in the key amino acid residues of the Replikin sequence, namely, lysine and histidine amino acid residues. The vaccine was engineered to inhibit the lethality of relatively lethal strains of ISAV.

The vaccine comprises a mixture of the following eighteen Replikin peptides in sterile water:

(SEQ ID NO: 1)
(1) HWKAAKYIK;
(SEQ ID NO: 2)
(2) KEAVNRGHWK;
(SEQ ID NO: 3)
(3) HKYNERLK;
(SEQ ID NO: 4)
(4) KGYIWKHK;
(SEQ ID NO: 5)
(5) KRMWDIGNKH;
(SEQ ID NO: 6)
(6) KLIDEVEVIKKKKH.
(SEQ ID NO: 7)
(7) HFRCMQGKQEVKGYIWK;
(SEQ ID NO: 8)
(8) KTVHWHLRVVK;
(SEQ ID NO: 9)
(9) KMTMMGKTVH;
(SEQ ID NO: 10)
(10) KMGDTRKEGYCH;
(SEQ ID NO: 11)
(11) KCWGMMFKTKSKMGDTRKEGYCH;
(SEQ ID NO: 12)
(12) HAIIFGKGEDKSGQNK;
(SEQ ID NO: 13)
(13) KVYGVLVDQLKLH;
(SEQ ID NO: 14)
(14) KLHGKDK;
(SEQ ID NO: 15)
(15) KLHGKDKVAGAKH;
(SEQ ID NO: 16)
(16) KDKVAGAKH;
(SEQ ID NO: 17)
(17) KQLHGQIHWK;
and
(SEQ ID NO: 18)
(18) KFESPREFRKGH.

Four pens of salmon are created. Pen 1 contains a control group, which is neither vaccinated nor inoculated with ISAV. Pen 2 contains a group that is vaccinated. Pen 3 contains a group that is vaccinated and inoculated with ISAV. Pen 4 contains a group that is not vaccinated but is nevertheless inoculated with ISAV.

The vaccine is administered to all salmon in pens 2 and 3 on days 7, 14, and 21. All salmon in pens 3 and 4 are inoculated with ISAV on day 28. Thereafter, the salmon are monitored for symptoms of ISAV infection. External body fluids are also tested via PCR for shedding of ISAV. The salmon in pen 2 demonstrate no symptoms of ISAV and shed no ISAV detected by PCR. The salmon in pen 4 demonstrate significant symptoms of ISAV and shed ISAV detected by PCR. The salmon in pen 3 demonstrate reduced symptoms of ISAV and shed considerably less ISAV detected by PCR than do the salmon in pen 4.

### Example 2

### ISAV Peak Replikin Concentrations Above 4.6 in Canada, Scotland, Norway, and Chile

pB1 gene area sequences for isolates of ISAV from Canada, Scotland, Norway, and Chile were queried at www.pubmed.com. Peak Replikin concentrations were identified above 4.6 Replikin sequences per 100 amino acid residues in Canada in 1997, 2005, 2006, 2007, 2008, and 2009. Peak Replikin concentrations above 4.6 were identified in Scotland in 1999. Peak Replikin concentrations above 4.6 were identified in Norway in 1998, 2000, 2004, 2005, 2008, and 2010. Peak Replikin concentrations above 4.6 were identified in Chile in 2004, 2007, and 2010.

Table 1 provides the data for Replikin concentrations above 4.6 in each country for each year.

TABLE 1

| | Canada | Scotland | Norway | Chile |
|---|---|---|---|---|
| 1997 | 4.6 | | | |
| 1998 | | | 5.6 | |
| 1999 | | 5.0 | | |
| 2000 | | | 5.5 | |
| 2001 | | | | |
| 2002 | | | | |
| 2003 | | | | |
| 2004 | | | 5.2 | 5.2 |
| 2005 | 5.2 | | 5.2 | |
| 2006 | 19.8 | | | |
| 2007 | 22.6 | | | 5.6 |
| 2008 | 5.0 | | 7.0 | |
| 2009 | 19.8 | | | |
| 2010 | | | 5.2 | 5.2 |

### Example 3

### Differentiation of Relative Lethality Among Different Isolates of ISAV

The relative lethality of different isolates of ISAV is determined by comparing the Replikin concentration of the pB1 gene area of each isolate of ISAV. Isolates are ranked for relative lethality based on the Replikin concentration. Isolates having higher Replikin concentration in the pB1 gene area are determined to have higher relative lethality and isolates having lower Replikin concentration in the pB1 gene area are determined to have relatively lower lethality.

The protein or genomic sequence of the polymerase protein was obtained from PubMed. The concentration of Replikin sequences in the pB1 gene area was determined for each isolate. The following accession numbers were analyzed to determine the Replikin concentration of various ISAV isolates from 2009: ACG50822, ACZ67857, ACZ67860, ACC77811, ABR45822, ABR45819, ABR45821, ABR45825, ABR45828. The Replikin concentrations for the pB1 gene area in these accession numbers for these isolates from 2009 were determined to be, respectively: 0.3, 0.9, 1.0, 1.5, 1.9, 2.5, 3.7, 19.8, and 19.8. The relative lethality of the isolates was then ranked by Replikin concentration from least lethal to most lethal as ACG50822<ACZ67857<ACZ67860<ACC77811<ABR45822<ABR45819<ABR45821<ABR45825=ABR45828.

### Example 4

### Replikin Concentration by Year for ISAV Isolates from Norway

The applicants reviewed publicly available pB1 gene area sequences for isolates of ISAV from Norway from 1997 through 2012 at www.pubmed.com. The data is provided in Table 2 below and is illustrated in FIG. 1.

TABLE 2
ISAV Annual Mean Replikin Concentration
in pB1 Gene Area in Isolates from Norway

| Year | Mean | Standard Deviation | Number of Isolates |
|---|---|---|---|
| 1997 | 2.6 | 1.8 | 2 |
| 1998 | 3.4 | 2.2 | 3 |
| 1999 | | | |
| 2000 | 0.3 | 0 | 1 |
| 2001 | 0.3 | 0.2 | 34 |
| 2002 | 1.6 | 0.4 | 38 |
| 2003 | 0.3 | 0.1 | 7 |
| 2004 | 3.7 | 0 | 1 |
| 2005 | 1.3 | 0.8 | 61 |
| 2006 | 0.4 | 0.2 | 45 |
| 2007 | | | |
| 2008 | 1.8 | 1.6 | 210 |
| 2009 | | | |
| 2010 | 2.2 | 1.6 | 10 |
| 2011 | 0.9 | 0.5 | 145 |
| 2012 | 0.5 | 0.2 | 8 |

### Example 5

### Peptide Sequences Conserved in ISAV pB1 Polymerase Geographic Regions

Table 3 provides examples of Replikin peptides that have been identified as conserved in isolates of ISAV pB1 across different countries and provides common positions for the beginning of the Replikin peptide within publicly disclosed protein sequences.

TABLE 3
Sequences Identified as Conserved across Countries

| | Common positions of | | | |
|---|---|---|---|---|
| | first amino acid of | | | |
| | Replikin sequence in | | | |
| | pB1 of ISAV (not | Conserved | Conserved | Conserved |
| Conserved Replikin Sequences | exclusive) | in Canada | in Norway | in Chile |
| HWKAAKYIK (SEQ ID NO: 1) | 587 and 600 | Yes | Yes | Yes |
| KEAVNRGHWK (SEQ ID NO: 2) | 580 and 593 | Yes | Yes | Yes |
| HKYNERLK (SEQ ID NO: 3) | 177 and 190 | Yes | Yes | Yes |
| KGYIWKHK (SEQ ID NO: 4) | 93, 171, and 184 | Yes | Yes | Yes |
| KRMWDIGNKH (SEQ ID NO: 5) | 56 and 69 | | Yes | Yes |
| KLIDEVEVIKKKKH (SEQ ID NO: 6) | 694 | | | Yes |
| HFRCMQGKQEVKGYIWK (SEQ ID NO: 7) | 173 | | | Yes |

### Example 6

### Conservation of SEQ ID NO: 1 in ISAV Isolates from Canada, Norway, and Chile

The sequence HWKAAKYIK (SEQ ID NO: 1) was identified in isolates of ISAV from Canada, Norway, and Chile. Conservation of Replikin sequences related to lethality in ISAV provides targets for vaccines against lethal isolates. SEQ ID NO: 1 and its homologues are a useful target for a vaccine against lethal ISAV.

In isolates from Canada from 2000 through 2011, SEQ ID NO: 1 was identified in the isolates reported at the following accession numbers at the identified sequence position:

| | |
|---|---|
| 2000 | AAF72700 position 587, AAF72699 position |
| | 587, AAF72698 position 587, AAF72697 |
| | position 587, AAF72696 position 587, |
| | AAF72695 position 587, AAF72694 position |
| | 587, AAF72693 position 587, AAF72692 |
| | position 587, AAF72691 position 587 |
| 2001 | AAK48525 position 509 |
| 2002 | AAL67962 position 600 |
| 2004 | YP_145804 position 600 |
| 2005 | ACV71155 position 600 |
| 2007 | ACB45380 position 600, ADR31788 position |
| | 99 |
| 2008 | ABG81414 position 600, ABF68026 position |
| | 600, ABF68025 position 600 |
| 2011 | Q8V3T6 position 600 |

In isolates from Norway from 1999 through 2010, SEQ ID NO: 1 was identified in the isolates reported at the following accession numbers at the identified sequence position:

| | |
|---|---|
| 1998 | CAA05486 position 600 |
| 2000 | AAF72699 position 587, AAF72698 position |
| | 587, AAF72697 position 587, AAF72696 |
| | position 587, AAF72695 position 587, |
| | AAF72694 position 587, AAF72692 position |
| | 587, AAF72691 position 587, AAF72700 |
| | position 587, AAF72693 position 587 |
| 2004 | ACJ37398 position 600 |
| 2005 | AAW72721 position 600, ACV71155 position |
| | 600 |
| 2008 | ABW93483 position 600, ABG65768 position |
| | 600, ABG65767 position 600, ABG65766 |
| | position 600, ABG65765 position 600, |
| | ABG65763 position 600, ABG65762 position |
| | 600, ABG65761 position 600, ABG65760 |
| | position 600, ABG65759 position 600, |
| | ABG65758 position 600, ABG65757 position |
| | 600, ABG65756 position 600, ABG65755 |
| | position 600, ACJ37394 position 558 |
| 2010 | ADR77506 position 600 |

In isolates from Chile from 2001 through 2010, SEQ ID NO: 1 was identified in the isolates reported at the following accession numbers at the identified positions in the sequence:

| | |
|---|---|
| 2001 | AAK48525 position 509 |
| 2004 | ACJ37398 position 600. |
| 2007 | ACB45380 position 600, ADR31788 position |
| | 99 |
| 2008 | ACJ37394 position 558 |
| 2010 | ADF36506 position 600, ADF36496 position |
| | 600 |

### Example 7

### Conservation of SEQ ID NO: 2 in ISAV Isolates from Canada, Norway, and Chile

The sequence KEAVNRGHWK (SEQ ID NO: 2) was identified in isolates of ISAV from Canada, Norway, and Chile.

In isolates from Canada from 2000 through 2011, SEQ ID NO: 2 was identified in the isolates reported at the following accession numbers at the identified positions in the sequence:

| | |
|---|---|
| 2000 | AAF72700 position 580, AAF72699 position |
| | 580, AAF72698 position 580, AAF72697 |
| | position 580, AAF72696 position 580, |
| | AAF72695 position 580, AAF72694 position |
| | 580, AAF72693 position 580, AAF72692 |
| | position 580, AAF72691 position 580. |
| 2001 | AAK48525 position 502 |
| 2002 | AAL67962 position 593 |
| 2004 | YP_145804 position 593 |
| 2005 | ACV71155 position 593. |
| 2007 | ACB45380 position 593 |
| 2008 | ABG81414 position 593, ABF68026 position |
| | 593, ABF68025 position 593 |
| 2011 | Q8V3T6 position 593 |

In isolates from Norway from 1998 through 2010, SEQ ID NO: 2 was identified in the isolates reported at the following accession numbers at the identified positions in the sequence:

| | |
|---|---|
| 1998 | CAA05486 position 593 |
| 2000 | AAF72699 position 580, AAF72698 position |
| | 580, AAF72697 position 580, AAF72696 |
| | position 580, AAF72695 position 580, |
| | AAF72694 position 580, AAF72692 position |
| | 580, AAF72691 position 580, AAF72700 |
| | position 580, AAF72693 position 580 |
| 2004 | ACJ37398 position 593 |
| 2005 | AAW72721 position 593, ACV71155 position |
| | 593 |
| 2008 | ABW93483 position 593, ABG65768 position |
| | 593, ABG65767 position 593, ABG65766 |
| | position 593, ABG65765 position 593, |
| | ABG65764 position 593, ABG65763 position |
| | 593, ABG65762 position 593, ABG65761 |
| | position 593, ABG65760 position 593, |
| | ABG65759 position 593, ABG65758 position |
| | 593, ABG65757 position 593, ABG65756 |
| | position 593, ABG65755 position 593, |
| | ACJ37394 position 551 |
| 2010 | ADR77506 position 593 |

In isolates from Chile from 2001 through 2010, SEQ ID NO: 2 was identified in the isolates reported at the following accession numbers at the identified positions in the sequence:

| | |
|---|---|
| 2001 | AAK48525 position 502 |
| 2004 | ACJ37398 position 593 |
| 2007 | ACB45380 position 593 |
| 2008 | ACJ37394 position 551 |
| 2010 | ADF36506 position 593, ADF36496 position |
| | 593 |

### Example 8

### Conservation of SEQ ID NO: 3 in ISAV Isolates from Canada, Norway, and Chile

The sequence HKYNERLK (SEQ ID NO: 3) was identified in isolates of ISAV from Canada, Norway, and Chile.

In isolates from Canada from 2000 through 2011, SEQ ID NO: 3 was identified in the isolates reported at the following accession numbers at the identified positions in the sequence:

| | |
|---|---|
| 2000 | AAF72700 position 177, AAF72699 position |
| | 177, AAF72698 position 177, AAF72697 |
| | position 177, AAF72696 position 177, |
| | AAF72695 position 177, AAF72694 position |
| | 177, AAF72693 position 177, AAF72692 |
| | position 177, AAF72691 position 177 |
| 2001 | AAK48525 position 99 |
| 2002 | AAL67962 position 190 |
| 2004 | YP_145804 position 190 |
| 2005 | ACV71155 position 190 |
| 2007 | ACB45380 position 190 |
| 2008 | ABG81414 position 190, ABF68026 position |
| | 190, ABF68025 position 190 |
| 2011 | Q8V3T6 position 190 |

In isolates from Norway from 1998 through 2010, SEQ ID NO: 3 was identified in the isolates reported at the following accession numbers at the identified positions in the sequence:

| | |
|---|---|
| 1998 | CAA05486 position 190 |
| 2000 | AAF72699 position 177, AAF72698 position |
| | 177, AAF72697 position 177, AAF72696 |
| | position 177, AAF72695 position 177, |
| | AAF72694 position 177, AAF72692 position |
| | 177, AAF72691 position 177, AAF72700 |
| | position 177, AAF72693 position 177 |
| 2004 | ACJ37398 position 190 |
| 2005 | AAW72721 position 190, ACV71155 position |
| | 190 |
| 2008 | ABW93483 position 190, ABG65768 position |
| | 190, ABG65767 position 190, ABG65766 |
| | position 190, ABG65765 position 190, |
| | ABG65764 position 190, ABG65763 position |
| | 190, ABG65762 position 190, ABG65761 |
| | position 190, ABG65760 position 190, |
| | ABG65759 position 190, ABG65758 position |
| | 190, ABG65757 position 190, ABG65756 |
| | position 190, ABG65755 position 190, |
| | ACJ37394 position 148 |
| 2010 | ADR77506 position 190 |

In isolates from Chile from 2001 through 2010, SEQ ID NO: 3 was identified in the isolates reported at the following accession numbers at the identified positions in the sequence:

| | |
|---|---|
| 2001 | AAK48525 position 99 |
| 2004 | ACJ37398 position 190 |
| 2007 | ACB45380 position 190 |
| 2008 | ACJ37394 position 148 |
| 2010 | ADF36506 position 190, |
| | ADF36496 position 190 |

### Example 9

### Conservation of SEQ ID NO: 4 in ISAV Isolates from Canada, Norway, and Chile

The sequence KGYIWKHK (SEQ ID NO: 4) was identified in isolates of ISAV from Canada, Norway, and Chile.

In isolates from Canada from 2000 through 2011, SEQ ID NO: 4 was identified in the isolates reported at the following accession numbers at the identified positions in the sequence:

| | |
|---|---|
| 2000 | AAF72700 position 171, AAF72699 position |
| | 171, AAF72698 position 171, AAF72697 |
| | position 171, AAF72696 position 171, |
| | AAF72695 position 171, AAF72694 position |
| | 171, AAF72693 position 171, AAF72692 |
| | position 171, AAF72691 position 171 |
| 2001 | AAK48525 position 93 |
| 2002 | AAL67962 position 184 |
| 2004 | YP_145804 position 184 |
| 2005 | ACV71155 position 184 |
| 2007 | ACB45380 position 184 |
| 2008 | ABG81414 position 184, ABF68025 position |
| | 184 |
| 2011 | Q8V3T6 position 184 |

In isolates from Norway from 1998 through 2010, SEQ ID NO: 4 was identified in the isolates reported at the following accession numbers at the identified positions in the sequence:

| | |
|---|---|
| 1998 | CAA05486 position 184 |
| 2000 | AAF72699 position 171, AAF72698 position |
| | 171, AAF72697 position 171, AAF72696 |
| | position 171, AAF72695 position 171, |
| | AAF72694 position 171, AAF72692 position |
| | 171, AAF72691 position 171, AAF72700 |
| | position 171, AAF72693 position 171 |
| 2004 | ACJ37398 position 184 |
| 2005 | AAW72721 position 184, ACV71155 position |
| | 184 |
| 2008 | ABW93483 position 184, ABG65768 position |
| | 184, ABG65767 position 184, ABG65766 |
| | position 184, ABG65765 position 184, |
| | ABG65764 position 184, ABG65763 position |
| | 184, ABG65762 position 184, ABG65761 |
| | position 184, ABG65760 position 184, |
| | ABG65759 position 184, ABG65758 position |
| | 184, ABG65757 position 184, ABG65756 |
| | position 184, ABG65755 position 184, |
| | ACJ37394 position 142 |
| 2010 | ADR77506 position 184 |

In isolates from Chile from 2001 through 2010, SEQ ID NO: 4 was identified in the isolates reported at the following accession numbers at the identified positions in the sequence:

| | |
|---|---|
| 2001 | AAK48525 position 93 |
| 2004 | ACJ37398 position 184 |
| 2007 | ACB45380 position 184 |
| 2008 | ACJ37394 position 142 |
| 2010 | ADF36506 position 184, ADF36496 position |
| | 184 |

### Example 10

### Conservation of SEQ ID NO: 5 in ISAV Isolates from Norway and Chile

The sequence KRMWDIGNKH (SEQ ID NO: 5) was identified in isolates of ISAV from Norway and Chile.

In isolates from Norway, SEQ ID NO: 5 was identified as conserved from 1998 through 2010 reported at the following accession numbers at the identified positions in the sequence:

| | |
|---|---|
| 1998 | CAA05486 position 69 |
| 2000 | AAF72699 position 56, AAF72698 position 56, |
| | AAF72697 position 56, AAF72696 position |
| | 56, AAF72695 position 56, AAF72694 |
| | position 56, AAF72692 position 56, |
| | AAF72691 position 56, AAF72693 position |
| | 56 |
| 2004 | ACJ37398 position 69 |
| 2005 | AAW72721 position 69, ACV71155 position |
| | 69 |
| 2008 | ABW93483 position 69, ABG65768 position |
| | 69, ABG65767 position 69, ABG65766 |
| | position 69, ABG65765 position 69, |
| | ABG65764 position 69, ABG65763 position |
| | 69, ABG65762 position 69, ABG65761 |
| | position 69, ABG65760 position 69, |
| | ABG65759 position 69, ABG65758 position |
| | 69, ABG65757 position 69, ABG65756 |
| | position 69, ABG65755 position 69, |
| | ACJ37394 position 27 |
| 2010 | ADR77506 position 69 |

In isolates from Chile, SEQ ID NO: 5 was identified as conserved from 2004 through 2010 reported at the following accession numbers at the identified positions in the sequence:

| | |
|---|---|
| 2004 | ACJ37398 position 69 |
| 2007 | ACB45380 position 69, ADR31787 position |
| | 69 |
| 2008 | ACJ37394 position 27 |
| 2010 | ADF36506 position 69, ADF36496 position 69 |

### Example 11

### Conservation of SEQ ID NO: 6 in ISAV Isolates from Chile

The sequence KLIDEVEVIKKKKH (SEQ ID NO: 6) was identified as conserved in isolates of ISAV from Chile from 2004 through 2010. SEQ ID NO: 6 was identified in the isolates reported at the following accession numbers at the identified positions in the sequence:

| | |
|---|---|
| 2004 | ACJ37398 position 694 |
| 2007 | ADR31788 position 193 |
| 2010 | ADF36496 position 694 |

### Example 12

### Conservation of SEQ ID NO: 7 in ISAV Isolates from Chile

The sequence HFRCMQGKQEVKGYIWK (SEQ ID NO: 7) was identified as conserved in isolates of ISAV from Chile from 2004 through 2010. SEQ ID NO: 7 was identified in the isolates reported at the following accession numbers at the identified positions in the sequence:

| | |
|---|---|
| 2004 | ACJ37398 position 173 |
| 2007 | ACB45380 position 173 |
| 2008 | ACJ37394 position 131 |
| 2010 | ADF36506 position 173, ADF36496 position173 |

### Example 13

### Conservation of SEQ ID NO: 8 in ISAV Isolates

The sequence KTVHWHLRVVK (SEQ ID NO: 8) was identified as conserved in isolates of ISAV from Canada from 2001 through 2011. SEQ ID NO: 8 was identified in the isolates reported at the following accession numbers at the identified positions in the sequence:

| | |
|---|---|
| 2001 | AAK97305 position 434 |
| 2002 | AAL67960 position 434 |
| 2004 | YP_145802 position 434 |
| 2008 | ABF68032 position 434, ABF68031 position |
| | 434, ABF68029 position 434, ABF68028 |
| | position 434, ABF68027 position 434, |
| | ABF68030 position 434 |
| 2011 | Q8V3T8 position 434 |

### Example 14

### Conservation of SEQ ID NO: 9 in ISAV Isolates

The sequence KMTMMGKTVH (SEQ ID NO: 9) was identified as conserved in isolates of ISAV from Canada from 2001 through 2011. SEQ ID NO: 9 was identified in the isolates reported at the following accession numbers at the identified positions in the sequence:

| | |
|---|---|
| 2001 | AAK97305 position 428 |
| 2002 | AAL67960 position 428 |
| 2004 | YP_145802 position 428 |
| 2008 | ABF68032 position 428, ABF68031 position |
| | 428, ABF68029 position 428, ABF68028 |
| | position 428, ABF68027 position 428, |
| | ABF68030 position 428 |
| 2011 | Q8V3T8 position 428 |

### Example 15

### Conservation of SEQ ID NO: 10 in ISAV Isolates

The sequence KMGDTRKEGYCH (SEQ ID NO: 10) was identified as conserved in isolates of ISAV from Canada from 1997 through 2011. SEQ ID NO: 10 was identified in the isolates reported at the following accession numbers at the identified positions in the sequence:

| | |
|---|---|
| 1997 | ACT83605 position 402 |
| 2001 | AAK97305 position 402 |
| 2002 | AAL67960 position 402 |
| 2004 | YP_145802 position 402 |
| 2008 | ABF68032 position 402, ABF68031 position |
| | 402, ABF68029 position 402, ABF68028 |
| | position 402, ABF68027 position 402, |
| | ABF68030 position 402 |
| 2011 | Q8V3T8 position 391 |

### Example 16

### Conservation of SEQ ID NO: 11 in ISAV Isolates

The sequence KCWGMMFKTKSKMGDTRKEGYCH (SEQ ID NO: 11) was identified as conserved in isolates of ISAV from Canada from 1997 through 2011. SEQ ID NO: 11 was identified in the isolates reported at the following accession numbers at the identified positions in the sequence:

| | |
|---|---|
| 1997 | ACT83605 position 391 |
| 2001 | AAK97305 position 391 |
| 2002 | AAL67960 position 391 |
| 2004 | YP_145802 position 391 |
| 2008 | ABF68032 position 391, ABF68031 position |
| | 391, ABF68029 position 391, ABF68028 |
| | position 391, ABF68027 position 391, |
| | ABF68030 position 391 |
| 2011 | Q8V3T8 position 391 |

### Example 17

### Conservation of SEQ ID NO: 12 in ISAV Isolates

The sequence HAIIFGKGEDKSGQNK (SEQ ID NO: 12) was identified as conserved in isolates of ISAV from at least Canada and Chile from 2001 through 2011. SEQ ID NO: 12 was identified in the isolates reported at the following accession numbers at the identified positions in the sequence:

| | |
|---|---|
| 2001 | AAK97305 position 413 |
| 2002 | AAL67960 position 413 |
| 2004 | YP_145802 position 413 |
| 2008 | ABF68032 position 413, ABF68031 position |
| | 413, ABF68029 position 413, ABF68028 |
| | position 413, ABF68027 position 413, |
| | ABF68030 position 413 |
| 2011 | Q8V3T8 position 413 |

### Example 18

### Conservation of SEQ ID NO: 13 in ISAV Isolates

The sequence KVYGVLVDQLKLH (SEQ ID NO: 13) was identified as conserved in isolates of ISAV from Canada from 1997 through 2009. SEQ ID NO: 13 was identified in the isolates reported at the following accession numbers at the identified positions in the sequence:

| | |
|---|---|
| 1997 | ADR31813 position 5 |
| 2002 | AAM11541 position 5, AAM11540 position 5, |
| | AAM11479 position 5, AAM11478 position |
| | 5, CAC80624 position 5 |
| 2004 | CAC80625 position 5 |
| 2005 | AAQ03075 position 5, AAQ03074 position 5 |
| 2006 | ABG21305 position 5 |
| 2007 | ABR13687 position 5, ABR13686 position 5, |
| | ABR13685 position 5, ABG72923 position 5, |
| | ABG72922 position 5, ABG72921 position 5, |
| | ADR31797 position 5, ABG72925 position 5, |
| | ABG72924 position 5. |
| 2009 | ABR45828 position 5, ABR45827 position 5, |
| | ABR45826 position 5, ABR45825 position 5, |
| | ABR45824 position 5, ABR45823 position 5, |
| | ABR45820 position 5, ABR45819 position 5, |
| | ABR45818 position 5, ABR45817 position 5, |
| | ABR45816 position 5, ABR45815 position 5, |
| | ABR45822 position 5, ABR45821 position 5 |

### Example 19

### Conservation of SEQ ID NO: 14 in ISAV Isolates

The sequence KLHGKDK (SEQ ID NO: 14) was identified as conserved in isolates of ISAV from Canada from 1997 through 2009. SEQ ID NO: 14 was identified in the isolates reported at the following accession numbers at the identified positions in the sequence:

| | |
|---|---|
| 1997 | ADR31813 position 15 |
| 2002 | AAM11541 position 15, AAM11540 position |
| | 15, AAM11479 position 15, AAM11478 |
| | position 15, CAC80624 position 15 |
| 2004 | CAC80625 position 15 |
| 2005 | AAQ03075 position 15, AAQ03074 position |
| | 15. |
| 2006 | ABG21305 position 15 |
| 2007 | ABR13687 position 15, ABR13686 position |
| | 15, ABR13685 position 15, ABG72923 |
| | position 15, ABG72922 position 15, |
| | ABG72921 position 15, ADR31797 position |
| | 15, ABG72925 position 15, ABG72924 |
| | position 15. |
| 2009 | ABR45828 position 15, ABR45827 position |
| | 15, ABR45826 position 15, ABR45820 |
| | position 15, ABR45819 position 15, |
| | ABR45818 position 15, ABR45817 position |
| | 15, ABR45816 position 15, ABR45815 |
| | position 15, ABR45822 position 15, |
| | ABR45821 position 15 |

### Example 20

### Conservation of SEQ ID NO: 15 in ISAV Isolates

The sequence KLHGKDKVAGAKH (SEQ ID NO: 15) was identified as conserved in isolates of ISAV from Canada from 2006 through 2009. SEQ ID NO: 15 was identified in the isolates reported at the following accession numbers at the identified positions in the sequence:

| | |
|---|---|
| 2006 | ABG21305 position 15 |
| 2007 | ABG72923 position 15 |
| 2009 | ABR45828 position 15, ABR45820 position |
| | 15, ABR45817 position 15 |

### Example 21

### Conservation of SEQ ID NO: 16 in ISAV Isolates

The sequence KDKVAGAKH (SEQ ID NO: 16) was identified as conserved in isolates of ISAV from Canada from 2006 through 2009. SEQ ID NO: 16 was identified in the isolates reported at the following accession numbers at the identified positions in the sequence:

| | |
|---|---|
| 2006 | ABG21305 position 19 |
| 2007 | ABG72923 position 19 |
| 2009 | ABR45828 position 19, ABR45820 position |
| | 19, ABR45817 position 19 |

### Example 22

### Conservation of SEQ ID NO: 17 in ISAV Isolates

The sequence KQLHGQIHWK (SEQ ID NO: 17) was identified as conserved in isolates of ISAV from Canada from 2006 through 2009. SEQ ID NO: 17 was identified in the isolates reported at the following accession numbers at the identified positions in the sequence:

| | |
|---|---|
| 2006 | ABG21305 position 37 |
| 2007 | ABR13687 position 37, ABG72923 position 37 |
| 2009 | ABR45828 position 37, ABR45825 position 37, |
| | ABR45820 position 37, ABR45817 position 37 |

### Example 23

### Conservation of SEQ ID NO: 18 in ISAV Isolates

The sequence KFESPREFRKGH (SEQ ID NO: 18) was identified as conserved in isolates of ISAV from Canada from 200 through 2011. SEQ ID NO: 18 was identified in the isolates reported at the following accession numbers at the identified positions in the sequence:

| | |
|---|---|
| 2000 | AAF72690 position 150 |
| 2001 | AAK51695 position 157, AAK51693 position |
| | 157, AAK51691 position 157 |
| 2002 | AAL67954 position 150, AAN63485 position |
| | 157. |
| 2003 | AAN74854 position 44, AAN74853 position |
| | 44, AAN74852 position 44, AAN74851 |
| | position 44, AAN74850 position 44, |
| | AAN74846 position 44, AAN74845 position |
| | 44 |
| 2006 | AAY52787 position 157, AAY52785 position |
| | 157, AAY52781 position 157, AAY52775 |
| | position 157, AAY52773 position 157. |
| 2011 | Q8V3U4 position 150 |

### Example 24

### Detailed Replikin Concentration Analysis for Each Accession Number in PubMed Listed as Sequences of an Isolate from the Canadian Province of Prince Edward Island (PEI)

PubMed was queried for a listing of all sequence data provided for isolates of ISAV from the Canadian province of Prince Edward Island. Replikin concentration for the sequence disclosed in each accession number was determined. The accession number, Replikin concentration (RC), source of sequence, serotype of ISAV (if published), and strain (if published) are provided in Table 4 below.

TABLE 4
Replikin concentration for various accession numbers reflecting
sequences of isolates of ISAV from Prince Edward Island

| Accession | Replikin | | | |
|---|---|---|---|---|
| Number | Concentration | Year | serotype | strain |
| ADR31811 | 1 | 1997 | unknown | NBISA01 |
| ADR31810 | 2.1 | 1997 | unknown | NBISA01 |
| ADR31815 | 2.4 | 1997 | unknown | NBISA01 |
| ACT83605 | 3.3 | 1997 | unknown | 485/9/97 |
| ADR31804 | 4.1 | 1997 | unknown | NBISA01 |
| ADR31813 | 4.2 | 1997 | unknown | NBISA01 |
| ADR31801 | 4.6 | 1997 | unknown | NBISA01 |
| AAK92417 | 0.3 | 2001 | unknown | 1490/98 |
| AAK92379 | 0.3 | 2001 | unknown | 301/98 |
| AAK92377 | 0.3 | 2001 | unknown | 810/9/99 |
| AAK92376 | 0.3 | 2001 | unknown | 835/9/98 |
| AAK91137 | 0.3 | 2001 | unknown | Bergen |
| AAK91148 | 0.3 | 2001 | unknown | U5575-1 |
| AAK91132 | 0.5 | 2001 | unknown | 390/98 |
| AAK92378 | 0.5 | 2001 | unknown | 485/9/97 |
| AAK92380 | 0.5 | 2001 | unknown | 832/98 |
| AAK92452 | 0.5 | 2001 | unknown | 912/99 |
| AAK91133 | 0.6 | 2001 | unknown | Glesvaer/2/90 |
| AAK48525 | 3.9 | 2001 | unknown | 7833-1 |
| AAQ81914 | 0.8 | 2003 | unknown | ISAV |
| AAQ81912 | 1 | 2003 | unknown | ISAV |
| AAQ81915 | 1.1 | 2003 | unknown | ISAV |
| AAQ81913 | 1.1 | 2003 | unknown | ISAV |
| YP_145807 | 0.8 | 2004 | unknown | ISAV |
| ACU21606 | 0.5 | 2005 | unknown | SK-05:90 |
| ACU21607 | 0.9 | 2005 | unknown | SK-05:90 |
| ACU21608 | 1.1 | 2005 | unknown | SK-05:144 |
| AAQ03075 | 1.9 | 2005 | unknown | ISAV |
| AAQ03080 | 2 | 2005 | unknown | ISAV |
| AAQ03074 | 2 | 2005 | unknown | ISAV |
| ACV71155 | 5.2 | 2005 | unknown | SK-05:90 |
| AAY29006 | 0.3 | 2006 | European | RPC/NB 04-085-1 |
| ABL09944 | 0.8 | 2006 | unknown | ILA149 |
| AAY52777 | 1.7 | 2006 | unknown | 390/98 |
| AAY52783 | 1.7 | 2006 | unknown | 485/9/97 |
| AAY85938 | 1.7 | 2006 | unknown | 810/9/99 |
| AAZ08562 | 1.7 | 2006 | unknown | ISAV |
| AAY52779 | 1.7 | 2006 | unknown | U5575-1 |
| AAY52785 | 2.5 | 2006 | unknown | RPC/NB 00-0593-1 |
| AAY52787 | 2.5 | 2006 | unknown | RPC/NB 01-0973-3 |
| AAY52781 | 2.5 | 2006 | unknown | RPC/NB 02-0775-14 |
| AAY52775 | 2.5 | 2006 | unknown | RPC/NB 02-1179-4 |
| AAY52773 | 2.5 | 2006 | unknown | RPC/NB 98-049-1 |
| AAY52774 | 3.1 | 2006 | unknown | RPC/NB 02-1179-4 |
| AAY52776 | 3.6 | 2006 | unknown | 390/98 |
| AAY52782 | 3.6 | 2006 | unknown | 485/9/97 |
| AAY85937 | 3.6 | 2006 | unknown | 810/9/99 |
| AAZ08561 | 3.6 | 2006 | unknown | ISAV |
| AAY52778 | 3.6 | 2006 | unknown | U5575-1 |
| AAY52784 | 4.6 | 2006 | unknown | RPC/NB 00-0593-1 |
| AAY52786 | 4.6 | 2006 | unknown | RPC/NB 01-0973-3 |
| AAY52780 | 4.6 | 2006 | unknown | RPC/NB 02-0775-14 |
| AAY52772 | 4.6 | 2006 | unknown | RPC/NB 98-049-1 |
| ABG21305 | 19.8 | 2006 | unknown | ISAV |
| ACG50825 | 0.3 | 2007 | European | VT11052007-27 |
| ACG56688 | 0.3 | 2007 | European | VT11282007-032 |
| ACG50824 | 0.3 | 2007 | European | VT11282007-033 |
| ACG56689 | 0.3 | 2007 | European | VT11282007-034 |
| ACG50826 | 0.3 | 2007 | European | VT11282007-037 tissue |
| ACG50828 | 0.3 | 2007 | European | VT11282007-040 |
| ACG50829 | 0.3 | 2007 | European | VT11282007-042 |
| ACG50830 | 0.3 | 2007 | European | VT11282007-043 |
| ACG50831 | 0.3 | 2007 | European | VT11282007-044 |
| ACG50816 | 0.3 | 2007 | European | VT11282007-35 |
| ACG50818 | 0.3 | 2007 | European | VT11282007-35 cell |
| | | | | culture |
| ACG56690 | 0.3 | 2007 | European | VT11282007-36 |
| ACG50819 | 0.3 | 2007 | European | VT11282007-36 |
| ACG50817 | 0.3 | 2007 | European | VT11282007-38 |
| ACG50827 | 0.3 | 2007 | European | VT11282007-38 cell |
| | | | | culture |
| ACG50823 | 0.3 | 2007 | European | VT11282007-39 |
| ADR31798 | 0.7 | 2007 | unknown | ADL-PM 3205 ISAV-07 |
| ABE02810 | 0.7 | 2007 | North American | ISAV |
| ABQ23338 | 1.1 | 2007 | unknown | 485/9/97 |
| ABE98325 | 1.1 | 2007 | North American | 7833-1 |
| ABQ23336 | 1.1 | 2007 | unknown | 810/9/99 |
| ACC99347 | 1.1 | 2007 | unknown | Biovac 24909 |
| ABE02812 | 1.1 | 2007 | North American | ISAV |
| ABE02811 | 1.1 | 2007 | North American | ISAV |
| ABE98323 | 1.1 | 2007 | North American | NBISA01 |
| ABE98326 | 1.1 | 2007 | North American | RPC/NB 01-0973-3 |
| ABE98324 | 1.1 | 2007 | North American | RPC/NB 02-1179-4 |
| ABR13688 | 1.1 | 2007 | unknown | RPC/NB 04-085-1 |
| ABE98322 | 1.1 | 2007 | North American | RPC/NB 98-049-1 |
| ABQ23337 | 1.1 | 2007 | unknown | U5575-1 |
| ACG56682 | 1.1 | 2007 | European | VT11282007-38 |
| ACG56683 | 1.1 | 2007 | European | VT11282007-39 |
| ADR31799 | 1.2 | 2007 | unknown | ADL-PM 3205 ISAV-07 |
| ADR31793 | 1.3 | 2007 | unknown | ADL-PM 3205 ISAV-07 |
| ACC77813 | 1.5 | 2007 | European | Biovac 26415-3 |
| ACC77810 | 1.5 | 2007 | European | Biovac 26572 |
| ABW96010 | 1.5 | 2007 | European | U24636 |
| ACG56680 | 1.5 | 2007 | European | VT11282007-37 |
| ABG72925 | 1.9 | 2007 | unknown | 485/9/97 |
| ADR31800 | 1.9 | 2007 | unknown | ADL-PM 3205 ISAV-07 |
| ABG72922 | 1.9 | 2007 | unknown | RPC/NB 02-0775-14 |
| ABR13686 | 1.9 | 2007 | unknown | RPC/NB 04-085-1 |
| ABG72921 | 2 | 2007 | unknown | RPC/NB 02-0775-14 |
| ADR31794 | 2.3 | 2007 | unknown | ADL-PM 3205 ISAV-07 |
| ADR31788 | 3.4 | 2007 | unknown | ADL-PM 3205 ISAV-07 |
| ABG72924 | 3.7 | 2007 | unknown | 485/9/97 |
| ADR31787 | 3.7 | 2007 | unknown | ADL-PM 3205 ISAV-07 |
| ABR13685 | 3.7 | 2007 | unknown | RPC/NB 04-085-1 |
| ADR31785 | 4.2 | 2007 | unknown | ADL-PM 3205 ISAV-07 |
| ACB45380 | 5.2 | 2007 | unknown | VT11152007-030 |
| ADR31797 | 5.6 | 2007 | unknown | ADL-PM 3205 ISAV-07 |
| ABG72923 | 19.8 | 2007 | unknown | RPC/NB 02-0775-14 |
| ABR13687 | 22.6 | 2007 | unknown | RPC/NB 04-085-1 |
| ACS94264 | 0.3 | 2008 | European | 13364-2006B |
| ACS94283 | 0.3 | 2008 | European | Biovac29560-2H |
| ACS94277 | 0.3 | 2008 | European | Biovac30740-3 |
| ACS94286 | 0.3 | 2008 | European | Biovac30741-8 |
| ACS94272 | 0.3 | 2008 | European | Biovac30942/943 |
| ACS94308 | 0.3 | 2008 | European | Biovac31587-8 |
| ACS94310 | 0.3 | 2008 | European | Biovac31587-9 |
| ACS94313 | 0.3 | 2008 | European | Biovac31588-14 |
| ACS94280 | 0.3 | 2008 | European | Biovac31589-16 |
| ACS94297 | 0.3 | 2008 | European | Biovac31589-17 |
| ACS94309 | 0.3 | 2008 | European | Biovac31590-18 |
| ACS94292 | 0.3 | 2008 | European | Biovac31590-20 |
| ACS94311 | 0.3 | 2008 | European | Biovac31591-6 |
| ACS94300 | 0.3 | 2008 | European | Biovac31591-7 |
| ACS94270 | 0.3 | 2008 | European | Biovac31592-2 |
| ACS94269 | 0.3 | 2008 | European | Biovac31592-4 |
| ACS94291 | 0.3 | 2008 | European | Biovac31647-3 |
| ACS94288 | 0.3 | 2008 | European | Biovac31647-8GH |
| ACS94314 | 0.3 | 2008 | European | Biovac31648-3GH |
| ACS94298 | 0.3 | 2008 | European | Biovac31648-5GH |
| ACS94304 | 0.3 | 2008 | European | Biovac31649-9 |
| ACS94285 | 0.3 | 2008 | European | Biovac31667-3GH |
| ACS94287 | 0.3 | 2008 | European | Biovac31667-5GH |
| ACS94307 | 0.3 | 2008 | European | Biovac31685-1 |
| ACS94290 | 0.3 | 2008 | European | Biovac31685-3 |
| ACS94266 | 0.3 | 2008 | European | Biovac31687-3 |
| ACS94267 | 0.3 | 2008 | European | Biovac31687-5 |
| ACS94279 | 0.3 | 2008 | European | Biovac31689-1 |
| ACS94278 | 0.3 | 2008 | European | Biovac31689-4 |
| ACS94284 | 0.3 | 2008 | European | Biovac31790-3GH |
| ACS94282 | 0.3 | 2008 | European | Biovac31790-9GH |
| ACS94301 | 0.3 | 2008 | European | Biovac31905-7Cz |
| ACS94293 | 0.3 | 2008 | European | Biovac31905-9Cz |
| ACS94281 | 0.3 | 2008 | European | Biovac32089-P1 |
| ACS94302 | 0.3 | 2008 | European | Biovac32232-2032LK |
| ACS94294 | 0.3 | 2008 | European | Biovac32232-2044K |
| ACS94276 | 0.3 | 2008 | European | Biovac32325-4 |
| ACS94305 | 0.3 | 2008 | European | Biovac32719-108 |
| ACS94303 | 0.3 | 2008 | European | Biovac32913-66 |
| ACS94295 | 0.3 | 2008 | European | Biovac32916-1 |
| ACS94274 | 0.3 | 2008 | European | Biovac32980-5 |
| ACS94296 | 0.3 | 2008 | European | Biovac33003-4 |
| ACS94312 | 0.3 | 2008 | European | Biovac33004-21 |
| ACS94289 | 0.3 | 2008 | European | Biovac33059-2 |
| ACS94299 | 0.3 | 2008 | European | Biovac33064-107 |
| ACS94275 | 0.3 | 2008 | European | PM-4165 #11 |
| ACS94306 | 0.3 | 2008 | European | PM-4165 #8 |
| ACG56686 | 0.3 | 2008 | European | VT04222008-106 |
| ACG56687 | 0.3 | 2008 | European | VT04222008-107 |
| ACG56691 | 0.3 | 2008 | European | VT05202008-111 |
| ACG56692 | 0.3 | 2008 | European | VT05202008-114 |
| ABF68024 | 0.6 | 2008 | unknown | 7833-1 |
| ABF68023 | 0.6 | 2008 | unknown | NBISA01 |
| ABF68035 | 0.6 | 2008 | unknown | RPC/NB 01-0973-3 |
| ABF68034 | 0.6 | 2008 | unknown | RPC/NB 02-1179-4 |
| ABF68033 | 0.6 | 2008 | unknown | RPC/NB 98-049-1 |
| ACT66013 | 0.7 | 2008 | European | Biovac 315924 |
| ACM17391 | 0.8 | 2008 | European | VT06132008-129 |
| ACM17392 | 0.9 | 2008 | European | Aquagestion 13492-9 |
| ACT66010 | 1.1 | 2008 | European | Biovac 2006B13364 |
| ACT66015 | 1.1 | 2008 | European | Biovac 30942/30943 |
| ACT66016 | 1.1 | 2008 | European | Biovac 316825 |
| ACT66009 | 1.2 | 2008 | European | Biovac 3168210 |
| ACT66018 | 1.5 | 2008 | European | Biovac 307403 |
| ACT66024 | 1.5 | 2008 | European | Biovac 307418 |
| ACT66039 | 1.5 | 2008 | European | Biovac 315878 |
| ACT66021 | 1.5 | 2008 | European | Biovac 32089P1 |
| ACT66036 | 1.5 | 2008 | European | Biovac 3291366 |
| ACT66017 | 1.5 | 2008 | European | PM4165 #11 |
| ACT66037 | 1.5 | 2008 | European | PM4165 #8 |
| ACG56679 | 1.5 | 2008 | European | VT04222008-106 |
| ACG56681 | 1.5 | 2008 | European | VT04222008-107 |
| ACG56684 | 1.5 | 2008 | European | VT05202008-111 |
| ACG56685 | 1.5 | 2008 | European | VT05202008-114 |
| ACT66029 | 1.6 | 2008 | European | Biovac 3159020 |
| ACT66041 | 1.6 | 2008 | European | Biovac 315916 |
| ACT66033 | 1.6 | 2008 | European | Biovac 315917 |
| ACT66028 | 1.6 | 2008 | European | Biovac 316473GH |
| ACT66042 | 1.6 | 2008 | European | Biovac 316483GH |
| ACT66032 | 1.6 | 2008 | European | Biovac 316485GH |
| ACT66038 | 1.6 | 2008 | European | Biovac 316851 |
| ACT66011 | 1.6 | 2008 | European | Biovac 316873 |
| ACT66012 | 1.6 | 2008 | European | Biovac 316875 |
| ACT66019 | 1.6 | 2008 | European | Biovac 316891 |
| ACT66020 | 1.6 | 2008 | European | Biovac 316894 |
| ACT66023 | 1.6 | 2008 | European | Biovac 317903GH |
| ACT66022 | 1.6 | 2008 | European | Biovac 317909GH |
| ACT66034 | 1.6 | 2008 | European | Biovac 319057Cz |
| ACT66030 | 1.6 | 2008 | European | Biovac 319059Cz |
| ACT66040 | 1.7 | 2008 | European | Biovac 3159018 |
| ACT66026 | 1.7 | 2008 | European | Biovac 316478GH |
| ACT66025 | 1.7 | 2008 | European | Biovac 316675GH |
| ACT66027 | 1.7 | 2008 | European | Biovac 316853 |
| ACT66035 | 1.7 | 2008 | European | Biovac 322322032LK |
| ACT66031 | 1.7 | 2008 | European | Biovac 322322044K |
| ACT66014 | 2.1 | 2008 | European | Biovac 315922 |
| ABF68026 | 4.2 | 2008 | unknown | RPC/NB 02-1179-4 |
| ABF68025 | 4.4 | 2008 | unknown | NBISA01 |
| ABG81414 | 4.4 | 2008 | North American | RPC/NB 02-0775-14 |
| ABF68030 | 5 | 2008 | unknown | 7833-1 |
| ABF68027 | 5 | 2008 | unknown | NBISA01 |
| ABF68031 | 5 | 2008 | unknown | RPC/NB 01-0593-1 |
| ABF68032 | 5 | 2008 | unknown | RPC/NB 01-0973-3 |
| ABF68029 | 5 | 2008 | unknown | RPC/NB 02-1179-4 |
| ABF68028 | 5 | 2008 | unknown | RPC/NB 98-049-1 |
| ACG50822 | 0.3 | 2009 | European | U-2611/VT01302008-068 |
| ACG50821 | 0.3 | 2009 | European | VT11152007-031 |
| ACG50820 | 0.3 | 2009 | European | VT11152007-30 |
| ACZ67857 | 0.9 | 2009 | unknown | 24984-1 |
| ACZ67853 | 0.9 | 2009 | unknown | 26416-6 |
| ACZ67854 | 0.9 | 2009 | unknown | 26905-10b |
| ACZ67855 | 0.9 | 2009 | unknown | 27102-1 |
| ACZ67856 | 0.9 | 2009 | unknown | 30735-2c |
| ACZ67859 | 0.9 | 2009 | unknown | 31807-2 |
| ACZ67861 | 0.9 | 2009 | unknown | 31991-3N |
| ACZ67860 | 1 | 2009 | unknown | 31903-3Br |
| ACZ67858 | 1.1 | 2009 | unknown | 26560-10b |
| ACZ67852 | 1.2 | 2009 | unknown | 32141 |
| ACZ67851 | 1.4 | 2009 | unknown | 29002 |
| ACC77811 | 1.5 | 2009 | European | Biovac 26829-2 |
| ACC77812 | 1.5 | 2009 | European | Biovac 26830 |
| ACZ67865 | 1.6 | 2009 | unknown | 31589-17 |
| ABR45822 | 1.9 | 2009 | unknown | 390/98 |
| ABR45824 | 1.9 | 2009 | unknown | RPC/NB 01-0593-1 |
| ABR45827 | 1.9 | 2009 | unknown | RPC/NB 01-0973-3 |
| ABR45816 | 1.9 | 2009 | unknown | RPC/NB 98-049-1 |
| ABR45823 | 2 | 2009 | unknown | RPC/NB 01-0593-1 |
| ABR45826 | 2 | 2009 | unknown | RPC/NB 01-0973-3 |
| ABR45818 | 2 | 2009 | unknown | RPC/NB 02-1179-4 |
| ABR45815 | 2 | 2009 | unknown | RPC/NB 98-049-1 |
| ABR45819 | 2.5 | 2009 | unknown | RPC/NB 02-1179-4 |
| ABR45821 | 3.7 | 2009 | unknown | 390/98 |
| ABR45825 | 19.8 | 2009 | unknown | RPC/NB 01-0593-1 |
| ABR45828 | 19.8 | 2009 | unknown | RPC/NB 01-0973-3 |
| ABR45820 | 19.8 | 2009 | unknown | RPC/NB 02-1179-4 |
| ABR45817 | 19.8 | 2009 | unknown | RPC/NB 98-049-1 |

### Example 25

### Replikin Concentration by Year for ISAV Isolates from Scotland

The applicants reviewed publicly available pB1 gene area sequences from isolates of ISAV from Scotland from 1998 through 2011 at www.pubmed.com. The data are provided in Table 5 below. The data are illustrated in FIG. 2.

TABLE 5
ISAV Annual Mean Replikin Concentration in
pB1 Gene Area in Isolates from Scotland

| | | Standard | Number of |
|---|---|---|---|
| Year | Mean | Deviation | Isolates |
| 1998 | 0.5 | 0 | 1 |
| 1999 | 5 | 3.9 | 3 |
| 2000 | | | |
| 2001 | 0.6 | 0.1 | 2 |
| 2002 | 2.4 | 1.8 | 2 |
| 2003 | 1.1 | 0.1 | 3 |
| 2004 | | | |
| 2005 | | | |
| 2006 | | | |
| 2007 | 0.3 | 0 | 1 |
| 2008 | | | |
| 2009 | | | |
| 2010 | | | |
| 2011 | 0.8 | 0 | 1 |

### Example 26

### Replikin Concentration by Year for ISAV Isolates from Chile

The applicants reviewed publicly available pB1 gene area sequences from isolates of ISAV from Chile from 2007 through 2010 at www.pubmed.com. The data are provided in Table 6 below. The data are illustrated in FIG. 3.

TABLE 6
ISAV Annual Mean Replikin Concentration
in pB1 Gene Area in Isolates from Chile

| | | Standard | Number of |
|---|---|---|---|
| Year | Mean | Deviation | Isolates |
| 2007 | 2.3 | 0 | 1 |
| 2008 | | | |
| 2009 | | | |
| 2010 | 2.4 | 1.5 | 19 |

### Example 27

### Replikin Concentration by Year for ISAV Isolates from Canada

The applicants reviewed publicly available pB1 gene area sequences from isolates of ISAV from Canada from 1997 through 2011 at www.pubmed.com. The data are provided in Table 7 below. The data are illustrated in FIG. 4.

TABLE 7
ISAV Annual Mean Replikin Concentration
in pB1 Gene Area in Isolates from Canada

| | | Standard | Number of |
|---|---|---|---|
| Year | Mean | Deviation | Isolates |
| 1997 | 3.1 | 1.4 | 6 |
| 1998 | 2.4 | 0 | 2 |
| 1999 | 3.6 | 2.6 | 4 |
| 2000 | | | |
| 2001 | 1.6 | 1.9 | 11 |
| 2002 | 2.4 | 1.7 | 18 |
| 2003 | 1.6 | 1.2 | 16 |
| 2004 | 1.9 | 1.7 | 13 |
| 2005 | 1.9 | 0.1 | 2 |
| 2006 | 2.9 | 3.6 | 27 |
| 2007 | 3.2 | 6 | 22 |
| 2008 | 3.6 | 2.4 | 15 |
| 2009 | 6.2 | 7.8 | 17 |
| 2010 | | | |
| 2011 | 2.3 | 1.9 | 8 |

What is claimed is:

1. An isolated or synthesized protein fragment or peptide comprising at least one peptide sequence that is at least 50% homologous with at least one Replikin peptide sequence identified in an infectious salmon anemia virus.

2. The isolated or synthesized protein fragment or peptide of claim 1, wherein said at least one peptide sequence is at least 80% homologous with at least one Replikin peptide sequence identified in an infectious salmon anemia virus.

3. The isolated or synthesized protein fragment or peptide of claim 1 comprising at least one Replikin peptide sequence identified in an infectious salmon anemia virus.

4. The isolated or synthesized protein fragment or peptide of claim 1 consisting essentially of at least one Replikin peptide sequence identified in an infectious salmon anemia virus or at least one peptide sequence that is at least 50% homologous with said at least one Replikin peptide sequence identified in an infectious salmon anemia virus.

5. The isolated or synthesized protein fragment or peptide of claim 1 consisting of at least one Replikin peptide sequence identified in an infectious salmon anemia virus or at least one peptide sequence that is at least 50% homologous with said at least one Replikin peptide sequence identified in an infectious salmon anemia virus.

6. The isolated or synthesized protein fragment or peptide of claim 1 comprising at least one Replikin peptide sequence of SEQ ID NO(s): 1-18 or at least one peptide sequence that is at least 50% homologous with SEQ ID NO(s): 1-18.

7. The isolated or synthesized protein fragment or peptide of claim 1 consisting essentially of at least one Replikin peptide sequence of SEQ ID NO(s): 1-18 or at least one peptide sequence that is at least 50% homologous with SEQ ID NO(s): 1-18.

8. The isolated or synthesized protein fragment or peptide of claim 1 consisting of at least one Replikin peptide sequence of SEQ ID NO(s): 1-18 or at least one peptide sequence that is at least 50% homologous with SEQ ID NO(s): 1-18.

9. A biosynthetic composition comprising the isolated or synthesized peptide of claim 1.

10. The biosynthetic composition of claim 9, wherein said isolated or synthesized peptide is chemically synthesized by solid phase methods.

11. An immunogenic and/or blocking composition comprising at least one protein, protein fragment, polypeptide, or peptide comprising at least one Replikin peptide identified in an infectious salmon anemia virus or at least one peptide sequence that is at least 50% homologous with at least one Replikin peptide identified in an infectious salmon anemia virus.

12. The immunogenic and/or blocking composition of claim 11 comprising at least one peptide sequence of SEQ ID NO(s): 1-18 and/or at least one peptide sequence that is at least 50% homologous with at least one peptide sequence of SEQ ID NO(s): 1-18.

13. A vaccine comprising the immunogenic composition of claim 11.

14. A vaccine comprising the immunogenic composition of claim 12.

15. The vaccine of claim 14 comprising a mixture of a plurality of peptide sequences of SEQ ID NO(s): 1-18 and/or a mixture of a plurality of peptide sequences that are at least 50% homologous with at least one of the peptide sequences of SEQ ID NO(s): 1-18.

16. A binding agent that preferentially binds to at least a portion of an amino acid sequence of at least one protein, protein fragment, polypeptide, or peptide that is at least 50% homologous with at least one Replikin peptide sequence identified in ISAV.

17. The binding agent of claim 16, wherein said portion of said amino acid sequence is at least one peptide sequence of SEQ ID NO(s): 1-18.

18. A method of making a vaccine comprising selecting at least one isolated or synthesized protein, protein fragment, polypeptide, or peptide comprising at least one peptide sequence that is at least 50% homologous with at least one Replikin peptide sequence identified in ISAV as a component of a vaccine; and making said vaccine.

19. A method of diagnosing a relatively more lethal form of infectious salmon anemia virus comprising determining the Replikin concentration of at least one portion of at least one protein of at least one isolate of ISAV or at least one portion of at least one gene that expresses at least one protein of the at least one isolate of ISAV and comparing the Replikin concentration of the at least one isolate of ISAV to a comparable Replikin concentration in at least one other isolate of ISAV.

20. The method of diagnosing a relatively more lethal form of infectious salmon anemia virus of claim 19, wherein said at least one portion of at least one protein comprises the entirety of at least one protein expressed in ISAV and the comparable Replikin concentration is the Replikin concentration of the entirety of the same protein expressed in ISAV from the at least one other isolate of ISAV and wherein the Replikin concentration of the more lethal isolate of ISAV is 4.6 per 100 amino acid residues or greater.
